SEQUENCE LISTING (105 sequences)

SEQ ID NO: 1 - protein, 15 aa, synthetic construct
  REGION 1..15: note = Description of Artificial Sequence: Synthetic peptide
GGGGSGGGGSGGGGS

SEQ ID NO: 2 - protein, 10 aa, synthetic construct
  REGION 1..10: note = Description of Artificial Sequence: Synthetic peptide
  VARIANT 2: note = MOD_RES - Phe or Asn
  VARIANT 3: note = MOD_RES - Thr, Glu or Met
  VARIANT 4: note = MOD_RES - Phe or Tyr
  VARIANT 6: note = MOD_RES - Lys, Thr, Gly, Asn or Glu
  VARIANT 8: note = MOD_RES - Ala or Pro
  VARIANT 9: note = MOD_RES - Met, Leu, Val or Ile
GXXXNXYXXN

SEQ ID NO: 3 - protein, 18 aa, synthetic construct
  REGION 1..18: note = Description of Artificial Sequence: Synthetic peptide
  VARIANT 5: note = MOD_RES - Lys or Gly
  VARIANT 6: note = MOD_RES - Tyr or Ser
  VARIANT 8: note = MOD_RES - Asn or Lys
  VARIANT 10: note = MOD_RES - Ala or Glu
  VARIANT 12: note = MOD_RES - Tyr or Glu
  VARIANT 14: note = MOD_RES - Ala or Lys
  VARIANT 16: note = MOD_RES - Ser, Glu, Asp, Ala or Gln
RIRSXXNXYXTXYXDXVK

SEQ ID NO: 4 - protein, 14 aa, synthetic construct
  REGION 1..14: note = Description of Artificial Sequence: Synthetic peptide
  VARIANT 2: note = MOD_RES - Gly, Ala or Thr
  VARIANT 5: note = MOD_RES - Gly or Asn
  VARIANT 6: note = MOD_RES - Asn or Asp
  VARIANT 8: note = MOD_RES - Tyr, His, Pro, Gln, Leu or Gly
  VARIANT 14: note = MOD_RES - Tyr or Thr
HXNFXXSXISYWAX

SEQ ID NO: 5 - protein, 14 aa, synthetic construct
  REGION 1..14: note = Description of Artificial Sequence: Synthetic peptide
  VARIANT 1: note = MOD_RES - Gly or Ala
  VARIANT 2: note = MOD_RES - Ser or Glu
  VARIANT 3: note = MOD_RES - Ser or Tyr
  VARIANT 4: note = MOD_RES - Thr, Phe, Lys or Ser
  VARIANT 6: note = MOD_RES - Ala or Tyr
  VARIANT 8: note = MOD_RES - Thr or Val
  VARIANT 9: note = MOD_RES - Ser, Asp, Lys, His or Val
  VARIANT 11: note = MOD_RES - Asn or Tyr
XXXXGXVXXGXYPN

SEQ ID NO: 6 - skipped

SEQ ID NO: 7 - protein, 9 aa, synthetic construct
  REGION 1..9: note = Description of Artificial Sequence: Synthetic peptide
  VARIANT 1: note = MOD_RES - Val, Thr or Ala
  VARIANT 5: note = MOD_RES - Ser, Asp or Ala
  VARIANT 7: note = MOD_RES - Arg or Ser
  VARIANT 9: note = MOD_RES - Val, Ile or Ala
XLWYXNXWX

SEQ ID NO: 8 - protein, 249 aa, synthetic construct
  REGION 1..249: note = Description of Artificial Sequence: Synthetic polypeptide
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 9 - protein, 249 aa, synthetic construct
  REGION 1..249: note = Description of Artificial Sequence: Synthetic polypeptide
EVQLVESGGGLVQPGGSLKLSCAASGFEFNKYAMNWVRQAPGKGLEWVARIRSKYNKYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSFGAVTSGNYPNWVQQKPGQAPRGLIGGTKFLAPGTPARFSGSLLGGKAALTLSGVQPEDEAEYYCVLWYDNRWVFGGGTKLTVL

SEQ ID NO: 10 - protein, 249 aa, synthetic construct
  REGION 1..249: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSHISYWAYWGQGTLVTVSSGGGGSGGGGSGGGGSQTVVTQEPSLTVSPGGTVTLTCGSSTGYVTSGNYPNWVQQKPGQAPRGLIGGTSFLAPGTPARFSGSLLGGKAALTLSGVQPEDEAEYYCVLWYSNRWIFGGGTKLTVL

SEQ ID NO: 11 - protein, 249 aa, synthetic construct
  REGION 1..249: note = Description of Artificial Sequence: Synthetic polypeptide
EVQLVESGGGLVQPGGSLKLSCAASGFMFNKYAMNWVRQAPGKGLEWVARIRSKSNNYATYYADSVKDRFTISRDDSKNTAYLQMNNLKTEDTAVYYCVRHGNFGNSYISYWATWGQGTLVTVSSGGGGSGGGGSGGGGSQTVVTQEPSLTVSPGGTVTLTCGSSFGAVTSGNYPNWVQQKPGQAPRGLIGGTKLLAPGTPARFSGSLLGGKAALTLSGVQPEDEAEYYCVLWYSNSWVFGGGTKLTVL

SEQ ID NO: 12 - protein, 249 aa, synthetic construct
  REGION 1..249: note = Description of Artificial Sequence: Synthetic polypeptide
EVQLVESGGGLVQPGGSLKLSCAASGFTFNTYAMNWVRQAPGKGLEWVARIRSKYNNYATYYKDSVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVVSGNYPNWVQQKPGQAPRGLIGGTEFLAPGTPARFSGSLLGGKAALTLSGVQPEDEAEYYCVLWYSNRWVFGGGTKLTVL

SEQ ID NO: 13 - protein, 249 aa, synthetic construct
  REGION 1..249: note = Description of Artificial Sequence: Synthetic polypeptide
EVQLVESGGGLVQPGGSLKLSCAASGFTYNKYAMNWVRQAPGKGLEWVARIRSKYNNYATYYADEVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSKGAVTSGNYPNWVQQKPGQAPRGLIGGTKELAPGTPARFSGSLLGGKAALTLSGVQPEDEAEYYCTLWYSNRWVFGGGTKLTVL

SEQ ID NO: 14 - protein, 249 aa, synthetic construct
  REGION 1..249: note = Description of Artificial Sequence: Synthetic polypeptide
EVQLVESGGGLVQPGGSLKLSCAASGNTFNKYAMNWVRQAPGKGLEWVARIRSKYNNYETYYADSVKDRFTISRDDSKNTAYLQMNNLKTEDTAVYYCVRHTNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTYFLAPGTPARFSGSLLGGKAALTLSGVQPEDEAEYYCVLWYSNRWVFGGGTKLTVL

SEQ ID NO: 15 - protein, 249 aa, synthetic construct
  REGION 1..249: note = Description of Artificial Sequence: Synthetic polypeptide
EVQLVESGGGLVQPGGSLKLSCAASGFTFNNYAMNWVRQAPGKGLEWVARIRSKYNNYATYYADAVKDRFTISRDDSKNTAYLQMNNLKTEDTAVYYCVRHGNFGNSQISYWAYWGQGTLVTVSSGGGGSGGGGSGGGGSQTVVTQEPSLTVSPGGTVTLTCGSSTGAVTDGNYPNWVQQKPGQAPRGLIGGIKFLAPGTPARFSGSLLGGKAALTLSGVQPEDEAEYYCVLWYSNRWVFGGGTKLTVL

SEQ ID NO: 16 - protein, 249 aa, synthetic construct
  REGION 1..249: note = Description of Artificial Sequence: Synthetic polypeptide
EVQLVESGGGLVQPGGSLKLSCAASGFTFNKYAVNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGESTGAVTSGNYPNWVQQKPGQAPRGLIGGTKILAPGTPARFSGSLLGGKAALTLSGVQPEDEAEYYCVLWYSNRWVFGGGTKLTVL

SEQ ID NO: 17 - protein, 249 aa, synthetic construct
  REGION 1..249: note = Description of Artificial Sequence: Synthetic polypeptide
EVQLVESGGGLVQPGGSLKLSCAASGFTFNKYPMNWVRQAPGKGLEWVARIRSKYNNYATYYADSVKDRFTISRDDSKNTAYLQMNNLKNEDTAVYYCVRHGNFNNSYISYWAYWGQGTLVTVSSGGGGSGGGGSGGGGSQTVVTQEPSLTVSPGGTVTLTCGSSTGAVTKGNYPNWVQQKPGQAPRGLIGGTKMLAPGTPARFSGSLLGGKAALTLSGVQPEDEAEYYCALWYSNRWVFGGGTKLTVL

SEQ ID NO: 18 - protein, 249 aa, synthetic construct
  REGION 1..249: note = Description of Artificial Sequence: Synthetic polypeptide
EVQLVESGGGLVQPGGSLKLSCAASGFTFNGYAMNWVRQAPGKGLEWVARIRSKYNNYATYYADEVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVVSGNYPNWVQQKPGQAPRGLIGGTEFLAPGTPARFSGSLLGGKAALTLSGVQPEDEAEYYCVLWYSNRWVFGGGTKLTVL

SEQ ID NO: 19 - protein, 249 aa, synthetic construct
  REGION 1..249: note = Description of Artificial Sequence: Synthetic polypeptide
EVQLVESGGGLVQPGGSLKLSCAASGNTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSSGGGGSGGGGSGGGGSQTVVTQEPSLTVSPGGTVTLTCGSSTGAVTHGNYPNWVQQKPGQAPRGLIGGTKVLAPGTPARFSGSLLGGKAALTLSGVQPEDEAEYYCVLWYSNRWVFGGGTKLTVL

SEQ ID NO: 20 - protein, 249 aa, synthetic construct
  REGION 1..249: note = Description of Artificial Sequence: Synthetic polypeptide
EVQLVESGGGLVQPGGSLKLSCAASGFTFNNYAMNWVRQAPGKGLEWVARIRSGYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSYTGAVTSGNYPNWVQQKPGQAPRGLIGGTKFNAPGTPARFSGSLLGGKAALTLSGVQPEDEAEYYCVLWYANRWVFGGGTKLTVL

SEQ ID NO: 21 - protein, 249 aa, synthetic construct
  REGION 1..249: note = Description of Artificial Sequence: Synthetic polypeptide
EVQLVESGGGLVQPGGSLKLSCAASGFEFNKYAMNWVRQAPGKGLEWVARIRSKYNNYETYYADSVKDRFTISRDDSKNTAYLQMNNLKTEDTAVYYCVRHGNFGNSLISYWAYWGQGTLVTVSSGGGGSGGGGSGGGGSQTVVTQEPSLTVSPGGTVTLTCGSSSGAVTSGNYPNWVQQKPGQAPRGLIGGTKFGAPGTPARFSGSLLGGKAALTLSGVQPEDEAEYYCVLWYSNRWVFGGGTKLTVL

SEQ ID NO: 22 - protein, 249 aa, synthetic construct
  REGION 1..249: note = Description of Artificial Sequence: Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 23 - protein, 10 aa, synthetic construct
  REGION 1..10: note = Description of Artificial Sequence: Synthetic peptide
GFTFNKYAMN

SEQ ID NO: 24 - protein, 18 aa, synthetic construct
  REGION 1..18: note = Description of Artificial Sequence: Synthetic peptide
RIRSKYNNYATYYADSVK

SEQ ID NO: 25 - protein, 14 aa, synthetic construct
  REGION 1..14: note = Description of Artificial Sequence: Synthetic peptide
HGNFGNSYISYWAY

SEQ ID NO: 26 - protein, 14 aa, synthetic construct
  REGION 1..14: note = Description of Artificial Sequence: Synthetic peptide
GSSTGAVTSGNYPN

SEQ ID NO: 27 - protein, 7 aa, synthetic construct
  REGION 1..7: note = Description of Artificial Sequence: Synthetic peptide
GTKFLAP

SEQ ID NO: 28 - protein, 9 aa, synthetic construct
  REGION 1..9: note = Description of Artificial Sequence: Synthetic peptide
VLWYSNRWV

SEQ ID NO: 29 - protein, 10 aa, synthetic construct
  REGION 1..10: note = Description of Artificial Sequence: Synthetic peptide
GNTFNKYAMN

SEQ ID NO: 30 - protein, 10 aa, synthetic construct
  REGION 1..10: note = Description of Artificial Sequence: Synthetic peptide
GFEFNKYAMN

SEQ ID NO: 31 - protein, 10 aa, synthetic construct
  REGION 1..10: note = Description of Artificial Sequence: Synthetic peptide
GFMFNKYAMN

SEQ ID NO: 32 - protein, 10 aa, synthetic construct
  REGION 1..10: note = Description of Artificial Sequence: Synthetic peptide
GFTYNKYAMN

SEQ ID NO: 33 - protein, 10 aa, synthetic construct
  REGION 1..10: note = Description of Artificial Sequence: Synthetic peptide
GFTFNNYAMN

SEQ ID NO: 34 - protein, 10 aa, synthetic construct
  REGION 1..10: note = Description of Artificial Sequence: Synthetic peptide
GFTFNGYAMN

SEQ ID NO: 35 - protein, 10 aa, synthetic construct
  REGION 1..10: note = Description of Artificial Sequence: Synthetic peptide
GFTFNTYAMN

SEQ ID NO: 36 - protein, 10 aa, synthetic construct
  REGION 1..10: note = Description of Artificial Sequence: Synthetic peptide
GFTFNEYAMN

SEQ ID NO: 37 - protein, 10 aa, synthetic construct
  REGION 1..10: note = Description of Artificial Sequence: Synthetic peptide
GFTFNKYPMN

SEQ ID NO: 38 - protein, 10 aa, synthetic construct
  REGION 1..10: note = Description of Artificial Sequence: Synthetic peptide
GFTFNKYAVN

SEQ ID NO: 39 - protein, 10 aa, synthetic construct
  REGION 1..10: note = Description of Artificial Sequence: Synthetic peptide
GFTFNKYAIN

SEQ ID NO: 40 - protein, 10 aa, synthetic construct
  REGION 1..10: note = Description of Artificial Sequence: Synthetic peptide
GFTFNKYALN

SEQ ID NO: 41 - protein, 18 aa, synthetic construct
  REGION 1..18: note = Description of Artificial Sequence: Synthetic peptide
RIRSGYNNYATYYADSVK

SEQ ID NO: 42 - protein, 18 aa, synthetic construct
  REGION 1..18: note = Description of Artificial Sequence: Synthetic peptide
RIRSKSNNYATYYADSVK

SEQ ID NO: 43 - protein, 18 aa, synthetic construct
  REGION 1..18: note = Description of Artificial Sequence: Synthetic peptide
RIRSKYNKYATYYADSVK

SEQ ID NO: 44 - protein, 18 aa, synthetic construct
  REGION 1..18: note = Description of Artificial Sequence: Synthetic peptide
RIRSKYNNYETYYADSVK

SEQ ID NO: 45 - protein, 18 aa, synthetic construct
  REGION 1..18: note = Description of Artificial Sequence: Synthetic peptide
RIRSKYNNYATEYADSVK

SEQ ID NO: 46 - protein, 18 aa, synthetic construct
  REGION 1..18: note = Description of Artificial Sequence: Synthetic peptide
RIRSKYNNYATYYKDSVK

SEQ ID NO: 47 - protein, 18 aa, synthetic construct
  REGION 1..18: note = Description of Artificial Sequence: Synthetic peptide
RIRSKYNNYATYYADEVK

SEQ ID NO: 48 - protein, 18 aa, synthetic construct
  REGION 1..18: note = Description of Artificial Sequence: Synthetic peptide
RIRSKYNNYATYYADAVK

SEQ ID NO: 49 - protein, 18 aa, synthetic construct
  REGION 1..18: note = Description of Artificial Sequence: Synthetic peptide
RIRSKYNNYATYYADQVK

SEQ ID NO: 50 - protein, 18 aa, synthetic construct
  REGION 1..18: note = Description of Artificial Sequence: Synthetic peptide
RIRSKYNNYATYYADDVK

SEQ ID NO: 51 - protein, 14 aa, synthetic construct
  REGION 1..14: note = Description of Artificial Sequence: Synthetic peptide
HANFGNSYISYWAY

SEQ ID NO: 52 - protein, 14 aa, synthetic construct
  REGION 1..14: note = Description of Artificial Sequence: Synthetic peptide
HTNFGNSYISYWAY

SEQ ID NO: 53 - protein, 14 aa, synthetic construct
  REGION 1..14: note = Description of Artificial Sequence: Synthetic peptide
HGNFNNSYISYWAY

SEQ ID NO: 54 - protein, 14 aa, synthetic construct
  REGION 1..14: note = Description of Artificial Sequence: Synthetic peptide
HGNFGDSYISYWAY

SEQ ID NO: 55 - protein, 14 aa, synthetic construct
  REGION 1..14: note = Description of Artificial Sequence: Synthetic peptide
HGNFGNSHISYWAY

SEQ ID NO: 56 - protein, 14 aa, synthetic construct
  REGION 1..14: note = Description of Artificial Sequence: Synthetic peptide
HGNFGNSPISYWAY

SEQ ID NO: 57 - protein, 14 aa, synthetic construct
  REGION 1..14: note = Description of Artificial Sequence: Synthetic peptide
HGNFGNSQISYWAY

SEQ ID NO: 58 - protein, 14 aa, synthetic construct
  REGION 1..14: note = Description of Artificial Sequence: Synthetic peptide
HGNFGNSLISYWAY

SEQ ID NO: 59 - protein, 14 aa, synthetic construct
  REGION 1..14: note = Description of Artificial Sequence: Synthetic peptide
HGNFGNSGISYWAY

SEQ ID NO: 60 - protein, 14 aa, synthetic construct
  REGION 1..14: note = Description of Artificial Sequence: Synthetic peptide
HGNFGNSYISYWAT

SEQ ID NO: 61 - protein, 14 aa, synthetic construct
  REGION 1..14: note = Description of Artificial Sequence: Synthetic peptide
ASSTGAVTSGNYPN

SEQ ID NO: 62 - protein, 14 aa, synthetic construct
  REGION 1..14: note = Description of Artificial Sequence: Synthetic peptide
GESTGAVTSGNYPN

SEQ ID NO: 63 - protein, 14 aa, synthetic construct
  REGION 1..14: note = Description of Artificial Sequence: Synthetic peptide
GSYTGAVTSGNYPN

SEQ ID NO: 64 - protein, 14 aa, synthetic construct
  REGION 1..14: note = Description of Artificial Sequence: Synthetic peptide
GSSFGAVTSGNYPN

SEQ ID NO: 65 - protein, 14 aa, synthetic construct
  REGION 1..14: note = Description of Artificial Sequence: Synthetic peptide
GSSKGAVTSGNYPN

SEQ ID NO: 66 - protein, 14 aa, synthetic construct
  REGION 1..14: note = Description of Artificial Sequence: Synthetic peptide
GSSSGAVTSGNYPN

SEQ ID NO: 67 - protein, 14 aa, synthetic construct
  REGION 1..14: note = Description of Artificial Sequence: Synthetic peptide
GSSTGYVTSGNYPN

SEQ ID NO: 68 - protein, 14 aa, synthetic construct
  REGION 1..14: note = Description of Artificial Sequence: Synthetic peptide
GSSTGAVVSGNYPN

SEQ ID NO: 69 - protein, 14 aa, synthetic construct
  REGION 1..14: note = Description of Artificial Sequence: Synthetic peptide
GSSTGAVTDGNYPN

SEQ ID NO: 70 - protein, 14 aa, synthetic construct
  REGION 1..14: note = Description of Artificial Sequence: Synthetic peptide
GSSTGAVTKGNYPN

SEQ ID NO: 71 - protein, 14 aa, synthetic construct
  REGION 1..14: note = Description of Artificial Sequence: Synthetic peptide
GSSTGAVTHGNYPN

SEQ ID NO: 72 - protein, 14 aa, synthetic construct
  REGION 1..14: note = Description of Artificial Sequence: Synthetic peptide
GSSTGAVTVGNYPN

SEQ ID NO: 73 - protein, 14 aa, synthetic construct
  REGION 1..14: note = Description of Artificial Sequence: Synthetic peptide
GSSTGAVTSGYYPN

SEQ ID NO: 74 - protein, 7 aa, synthetic construct
  REGION 1..7: note = Description of Artificial Sequence: Synthetic peptide
GIKFLAP

SEQ ID NO: 75 - protein, 7 aa, synthetic construct
  REGION 1..7: note = Description of Artificial Sequence: Synthetic peptide
GTEFLAP

SEQ ID NO: 76 - protein, 7 aa, synthetic construct
  REGION 1..7: note = Description of Artificial Sequence: Synthetic peptide
GTYFLAP

SEQ ID NO: 77 - protein, 7 aa, synthetic construct
  REGION 1..7: note = Description of Artificial Sequence: Synthetic peptide
GTSFLAP

SEQ ID NO: 78 - protein, 7 aa, synthetic construct
  REGION 1..7: note = Description of Artificial Sequence: Synthetic peptide
GTNFLAP

SEQ ID NO: 79 - protein, 7 aa, synthetic construct
  REGION 1..7: note = Description of Artificial Sequence: Synthetic peptide
GTKLLAP

SEQ ID NO: 80 - protein, 7 aa, synthetic construct
  REGION 1..7: note = Description of Artificial Sequence: Synthetic peptide
GTKELAP

SEQ ID NO: 81 - protein, 7 aa, synthetic construct
  REGION 1..7: note = Description of Artificial Sequence: Synthetic peptide
GTKILAP

SEQ ID NO: 82 - protein, 7 aa, synthetic construct
  REGION 1..7: note = Description of Artificial Sequence: Synthetic peptide
GTKMLAP

SEQ ID NO: 83 - protein, 7 aa, synthetic construct
  REGION 1..7: note = Description of Artificial Sequence: Synthetic peptide
GTKVLAP

SEQ ID NO: 84 - protein, 7 aa, synthetic construct
  REGION 1..7: note = Description of Artificial Sequence: Synthetic peptide
GTKFNAP

SEQ ID NO: 85 - protein, 7 aa, synthetic construct
  REGION 1..7: note = Description of Artificial Sequence: Synthetic peptide
GTKFGAP

SEQ ID NO: 86 - protein, 7 aa, synthetic construct
  REGION 1..7: note = Description of Artificial Sequence: Synthetic peptide
GTKFLVP

SEQ ID NO: 87 - protein, 9 aa, synthetic construct
  REGION 1..9: note = Description of Artificial Sequence: Synthetic peptide
TLWYSNRWV

SEQ ID NO: 88 - protein, 9 aa, synthetic construct
  REGION 1..9: note = Description of Artificial Sequence: Synthetic peptide
ALWYSNRWV

SEQ ID NO: 89 - protein, 9 aa, synthetic construct
  REGION 1..9: note = Description of Artificial Sequence: Synthetic peptide
VLWYDNRWV

SEQ ID NO: 90 - protein, 9 aa, synthetic construct
  REGION 1..9: note = Description of Artificial Sequence: Synthetic peptide
VLWYANRWV

SEQ ID NO: 91 - protein, 9 aa, synthetic construct
  REGION 1..9: note = Description of Artificial Sequence: Synthetic peptide
VLWYSNSWV

SEQ ID NO: 92 - protein, 9 aa, synthetic construct
  REGION 1..9: note = Description of Artificial Sequence: Synthetic peptide
VLWYSNRWI

SEQ ID NO: 93 - protein, 9 aa, synthetic construct
  REGION 1..9: note = Description of Artificial Sequence: Synthetic peptide
VLWYSNRWA

SEQ ID NO: 94 - protein, 249 aa, synthetic construct
  REGION 1..249: note = Description of Artificial Sequence: Synthetic polypeptide
EVQLVESGGGLVQPGGSLKLSCAASGFTFNKYALNWVRQAPGKGLEWVARIRSKYNNYATEYADSVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVTSGNYPNWVQQKPGQAPRGLIGGTNFLAPGTPERFSGSLLGGKAALTLSGVQPEDEAEYYCVLWYSNRWAFGGGTKLTVL

SEQ ID NO: 95 - protein, 249 aa, synthetic construct
  REGION 1..249: note = Description of Artificial Sequence: Synthetic polypeptide
EVQLVESGGGLVQPGGSLKLSCAASGFTFNEYAMNWVRQAPGKGLEWVARIRSKYNNYATYYADDVKDRFTISRDDSKNTAYLQMNNLKTEDTAVYYCVRHGNFGNSGISYWAYWGQGTLVTVSSGGGGSGGGGSGGGGSQTVVTQEPSLTVSPGGTVTLTCGSSTGAVTVGNYPNWVQQKPGQAPRGLIGGTEFLAPGTPARFSGSLLGGKAALTLSGVQPEDEAEYYCVLWYSNRWVFGGGTKLTVL

SEQ ID NO: 96 - protein, 20 aa, synthetic construct
  REGION 1..20: note = Description of Artificial Sequence: Synthetic peptide
  VARIANT 1..20: note = MISC_FEATURE - This sequence may encompass 1-10 "Gly Ser" repeating units
GSGSGSGSGSGSGSGSGSGS

SEQ ID NO: 97 - protein, 30 aa, synthetic construct
  REGION 1..30: note = Description of Artificial Sequence: Synthetic polypeptide
  VARIANT 1..30: note = MISC_FEATURE - This sequence may encompass 1-10 "Gly Gly Ser" repeating units
GGSGGSGGSGGSGGSGGSGGSGGSGGSGGS

SEQ ID NO: 98 - protein, 40 aa, synthetic construct
  REGION 1..40: note = Description of Artificial Sequence: Synthetic polypeptide
  VARIANT 1..40: note = MISC_FEATURE - This sequence may encompass 1-10 "Gly Gly Gly Ser" repeating units
GGGSGGGSGGGSGGGSGGGSGGGSGGGSGGGSGGGSGGGS

SEQ ID NO: 99 - protein, 40 aa, synthetic construct
  REGION 1..40: note = Description of Artificial Sequence: Synthetic polypeptide
  VARIANT 1..40: note = MISC_FEATURE - This sequence may encompass 1-10 "Gly Gly Ser Gly" repeating units
GGSGGGSGGGSGGGSGGGSGGGSGGGSGGGSGGGSGGGSG

SEQ ID NO: 100 - protein, 50 aa, synthetic construct
  REGION 1..50: note = Description of Artificial Sequence: Synthetic polypeptide
  VARIANT 1..50: note = MISC_FEATURE - This sequence may encompass 1-10 "Gly Gly Ser Gly Gly" repeating units
GGSGGGGSGGGGSGGGGSGGGGSGGGGSGGGGSGGGGSGGGGSGGGGSGG

SEQ ID NO: 101 - protein, 50 aa, synthetic construct
  REGION 1..50: note = Description of Artificial Sequence: Synthetic polypeptide
  VARIANT 1..50: note = MISC_FEATURE - This sequence may encompass 1-10 "Gly Gly Gly Gly Ser" repeating units
GGGGSGGGGSGGGGSGGGGSGGGGSGGGGSGGGGSGGGGSGGGGSGGGGS

SEQ ID NO: 102 - protein, 50 aa, synthetic construct
  REGION 1..50: note = Description of Artificial Sequence: Synthetic polypeptide
  VARIANT 1..50: note = MISC_FEATURE - This sequence may encompass 1-10 "Gly Gly Gly Gly Gly" repeating units
GGGGGGGGGGGGGGGGGGGGGGGGGGGGGGGGGGGGGGGGGGGGGGGGGG

SEQ ID NO: 103 - protein, 30 aa, synthetic construct
  REGION 1..30: note = Description of Artificial Sequence: Synthetic polypeptide
GGGGGGGGGGGGGGGGGGGGGGGGGGGGGG

SEQ ID NO: 104 - protein, 20 aa, synthetic construct
  REGION 1..20: note = Description of Artificial Sequence: Synthetic peptide
GGGGSGGGGSGGGGSGGGGS

SEQ ID NO: 105 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2024/0084009 A1
(43) Pub. Date: Mar. 14, 2024

(54) SINGLE CHAIN VARIABLE FRAGMENT CD3 BINDING PROTEINS

(71) Applicant: Harpoon Therapeutics, Inc., South San Francisco, CA (US)

(72) Inventors: Robert B. DUBRIDGE, Belmont, CA (US); Pui SETO, San Carlos, CA (US); Richard J. AUSTIN, San Francisco, CA (US); Luke EVNIN, San Francisco, CA (US); Jeanmarie GUENOT, San Francisco, CA (US); Bryan LEMON, Mountain View, CA (US)

(21) Appl. No.: 18/188,037

(22) Filed: Mar. 22, 2023

Related U.S. Application Data
(63) Continuation of application No. 16/773,806, filed on Jan. 27, 2020, now Pat. No. 11,623,958.
Application No. 16/773,806 is a continuation of application No. 15/704,620, filed on Sep. 14, 2017, now Pat. No. 10,066,016.
Application No. 15/704,620 is a continuation of application No. 15/600,264, filed on May 19, 2017, now Pat. No. 10,544,221.
(60) Provisional application No. 62/339,685, filed on May 20, 2016.

(51) Int. Cl. C07K 16/28; C07K 16/18

(52) U.S. Cl. CPC C07K 16/2809; C07K 16/18; C07K 16/2887; C07K 2317/31; C07K 2317/565; C07K 2317/622; C07K 2317/73; C07K 2317/92; C07K 2317/94

(57) ABSTRACT

Disclosed herein are single chain variable fragment CD3 binding proteins with improved binding affinities, and robust aggregation profiles. Also described are multispecific binding proteins comprising a single chain variable fragment CD3 binding protein according to the instant disclosure. Pharmaceutical compositions comprising the binding proteins disclosed herein and methods of using such formulations are provided.

### CROSS-REFERENCE

This is a continuation application of U.S. application Ser. No. 16/773,806, filed Jan. 27, 2020, which is a continuation application of U.S. application Ser. No. 15/704,620, filed Sep. 14, 2017 (now U.S. Pat. No. 10,066,016, issued Sept. 4, 2018), which is a continuation application of U.S. application Ser. No. 15/600,264, filed May 19, 2017 (now U.S. Pat. No. 10,544,221, issued Jan. 28, 2020), and claims the benefit of U.S. Provisional Application No. 62/339,685, filed May 20, 2016, all of which are incorporated by reference herein in their entirety.

### SEQUENCE LISTING

The instant application contains a Sequence Listing which has been submitted electronically in XML format and is hereby incorporated by reference in its entirety. Said XML copy, created on Apr. 28, 2023, is named 47517-704_303 SL._xml and is 153,393 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE INVENTION

CD3 is a homodimeric or heterodimeric antigen expressed on T cells in association with the T cell receptor complex (TCR) and is required for T cell activation. Anti-CD3 antibodies have therapeutic purposes involving the activation of T cells. Present disclosure provides single chain variable fragment CD3 binding proteins, including multispecific antibodies containing the same.

### SUMMARY OF THE INVENTION

In one embodiment is disclosed a single chain variable fragment CD3 binding protein, comprising a variable heavy chain region (VH), a variable light chain region (VL), and a linker, wherein VH comprises complementarity determining regions HC CDR1, HC CDR2, and HC CDR3, wherein VL comprises complementarity determining regions LC CDR1, LC CDR2, and LC CDR3, wherein (a) the amino acid sequence of HC CDR1 is as set forth in GX₁X₂X₃NX₄YX₅X₆N (SEQ ID NO. 2), X₁ is phenylalanine or asparagine, X₂ is threonine, glutamic acid or methionine, X₃ is phenylalanine or tyrosine, X₄ is lysine, threonine, glycine, aspragine or glutamic acid, X₅ is alanine or proline, X₆ is methionine, leucine, valine or isoleucine; (b) the amino acid sequence of HC CDR2 is as set forth in RIRSX₇X₈NX₉YX₁₀TX₁₁YX₁₂DX₁₃VK (SEQ ID NO. 3), X₇ is lysine or glycine, X₈ is tyrosine or serine, X₉ is asparagine or lysine, X₁₀ is alanine or glutamic acid, X₁₁ is tyrosine or glutamic acid, X₁₂ is alanine or lysine, X₁₃ is serine, glutamic acid, aspartic acid, alanine, or glutamine; (c) the amino acid sequence of HC CDR3 is as set forth in HX₁₄NFX₁₅ X₁₆SX₁₇ISYWAX₁₈ (SEQ ID NO. 4), X₁₄ is glycine, alanine, or threonine, X₁₅ is glycine or asparagine, X₁₆ is asparagine or aspartic acid, X₁₇ is tyrosine, histidine, proline, glutamine, leucine or glycine, X18 is tyrosine or threonine; (d) the amino acid sequence of LC CDR1 is as set forth in X₁₉X₂₀X₂₁X₂₂GX₂₃VX₂₄X₂₅GX₂₆YPN (SEQ ID NO. 5), X₁₉ is glycine or alanine, X₂₀ is serine or glutamic acid, X₂₁ is serine or tyrosine, X₂₂ is threonine, phenylalanine, lysine, or serine, X₂₃ is alanine or tyrosine, X₂₄ is threonine or valine, X25 is serine, aspartic acid, lysine, histidine or valine, X₂₆ asparagine or tyrosine; (e) the amino acid sequence of LC CDR2 is as set forth in GX₂₇X₂₈X₂₉X₃₀X₃₁P, X₂₇ is threonine or isoleucine, X₂₈ is lysine, glutamic acid, tyrosine, asparagine or serine, X₂₉ is phenylalanine, leucine, glutamic acid, isoleucine, methionine, or valine, X₃₀ is leucine, asparagine, or glycine, X₃₁ is alanine or valine; and (f) the amino acid sequence of LC CDR3 is as set forth in X₃₂LWYX₃₃NX₃₄WX₃₅ (SEQ ID NO. 7), X₃₂ is valine, threonine or alanine, X₃₃ is serine, aspartic acid or alanine, X₃₄ is arginine or serine, X₃₅ is valine, isoleucine or alanine, wherein X, X₂, X₃, X₄, X₅, X₆, X₇, X₈, X₉, X₁₀, X₁₁, X_{12,}X₁₃, X₁₄, X₁₅, X₁₆, X₁₇, X₁₈, X₁₉, X₂₀, X₂₁, X₂₂, X₂₃, X_{24,} X25, X26, X27, X₂₈, X₂₉, X₃₀, X₃₁, X₃₂, X₃₃, X₃₄ and X35 are not simultaneously phenylalanine, threonine, phenylalanine, lysine, alanine, methionine, lysine, tyrosine, asparagine, alanine, tyrosine, alanine, serine, glycine, glycine, asparagine, tyrosine, tyrosine, glycine, serine, serine, threonine, alanine, threonine, serine, asparagine, threonine, lysine, phenylalanine, leucine, alanine, valine, serine, arginine, and valine respectively.

In some embodiments, the single chain variable fragment CD3 binding protein comprises the following formula: f1-r1-f2-r2-f3-r3-f4-r4-f5-r5-f6-r6-f7, wherein, rl is SEQ ID NO: 2; r2 is SEQ ID NO: 3; r3 is SEQ ID NO: 4; r4 is SEQ ID NO:5; r5 is SEQ ID NO:6; and r6 is SEQ ID NO:7; and wherein f_{1,} f₂, f₃, f₄, and f₅ are framework residues selected so that said protein is at least eighty percent identical to the amino acid sequence set forth in SEQ ID NO: 22.

In some embodiments, the single chain variable fragment CD3 binding protein comprises an amino acid sequence wherein r1 comprises SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO.38, SEQ ID NO. 39, or SEQ ID NO. 40. In some embodiments, the single chain variable fragment CD3 binding protein comprises an amino acid sequence wherein r2 comprises SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48, SEQ ID NO. 49, or SEQ ID NO. 50. In some embodiments, the single chain variable fragment CD3 binding protein comprises an amino acid sequence wherein r3 comprises SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 50, or SEQ ID NO. 60. In some embodiments, the single chain variable fragment CD3 binding protein comprises an amino acid sequence wherein r4 comprises SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, or SEQ ID NO. 73 In some embodiments, the single chain variable fragment CD3 binding protein comprises an amino acid sequence wherein r5 comprises SEQ ID NO. 74, SEQ ID NO. 75, SEQ ID NO. 76, SEQ ID NO. 77, SEQ ID NO. 78, SEQ ID NO. 79, SEQ ID NO. 80, SEQ ID NO. 81, SEQ ID NO. 82, SEQ ID NO. 83, SEQ ID NO. 84, SEQ ID NO. 85, or SEQ ID NO. 86. In some embodiments, the single chain variable fragment CD3 binding protein comprises an amino acid sequence wherein r6 comprises SEQ ID NO. 87, SEQ ID NO. 88, SEQ ID NO. 89, SEQ ID NO. 90, SEQ ID NO. 91, SEQ ID NO. 92, or SEQ ID NO. 93. In some embodiments, the single chain variable fragment CD3 binding protein comprises an amino acid sequence wherein r1 is SEQ ID NO. 39, r2 is SEQ ID NO. 49, r3 is SEQ ID NO. 51, r4 is SEQ ID NO. 61, r5 is SEQ ID NO. 86, and r6 is SEQ ID NO. 87. In some embodiments, the single chain variable fragment CD3 binding protein comprises an amino acid sequence wherein r1 is SEQ ID NO. 30, r2 is SEQ ID NO. 43, r4 is SEQ ID NO. 64, and r6 is SEQ ID NO. 89. In some embodiments, the single chain variable fragment CD3 binding protein comprises an amino acid sequence wherein r3 is SEQ ID NO. 55, r4 is SEQ ID NO. 67, r5 is SEQ ID NO. 77, and r6 is SEQ ID NO. 92. In some embodiments, the single chain variable fragment CD3 binding protein comprises an amino acid sequence wherein r1 is SEQ ID NO. 31, r2 is SEQ ID NO. 42, r3 is SEQ ID NO. 60, r4 is SEQ ID NO. 64, r5 is SEQ ID NO. 79, and r6 is SEQ ID NO. 91. In some embodiments, the single chain variable fragment CD3 binding protein comprises an amino acid sequence wherein r1 is SEQ ID NO. 35, r2 is SEQ ID NO. 46, r3 is SEQ ID NO. 56, r4 is SEQ ID NO. 68, and r5 is SEQ ID NO. 75. In some embodiments, the single chain variable fragment CD3 binding protein comprises an amino acid sequence wherein r1 is SEQ ID NO. 32, r2 is SEQ ID NO. 47, r3 is SEQ ID NO. 56, r4 is SEQ ID NO. 65, r5 is SEQ ID NO. 80, and r6 is SEQ ID NO. 87. In some embodiments, the single chain variable fragment CD3 binding protein comprises an amino acid sequence wherein r1 is SEQ ID NO. 29, r2 is SEQ ID NO. 44, r3 is SEQ ID NO. 52, r4 is SEQ ID NO. 73, and r5 is SEQ ID NO. 76. In some embodiments, the single chain variable fragment CD3 binding protein comprises an amino acid sequence wherein r1 is SEQ ID NO. 33, r2 is SEQ ID NO. 48, r3 is SEQ ID NO. 57, r4 is SEQ ID NO. 69, and r5 is SEQ ID NO. 74. In some embodiments, the single chain variable fragment CD3 binding protein comprises an amino acid sequence wherein r1 is SEQ ID NO. 38, r4 is SEQ ID NO. 62, and r5 is SEQ ID NO. 81. In some embodiments, the single chain variable fragment CD3 binding protein comprises an amino acid sequence wherein r1 is SEQ ID NO. 37, r3 is SEQ ID NO. 53, r4 is SEQ ID NO. 70, r5 is SEQ ID NO. 82, and r6 is SEQ ID NO. 88. In some embodiments, the single chain variable fragment CD3 binding protein comprises an amino acid sequence wherein r1 is SEQ ID NO. 34, r2 is SEQ ID NO. 47, r3 is SEQ ID NO. 56, r4 is SEQ ID NO. 68 and r5 is SEQ ID NO. 75. In some embodiments, the single chain variable fragment CD3 binding protein comprises an amino acid sequence wherein r1 is SEQ ID NO. 29, r3 is SEQ ID NO. 54, r4 is SEQ ID NO. 71 and r5 is SEQ ID NO. 83. In some embodiments, the single chain variable fragment CD3 binding protein comprises an amino acid sequence wherein r1 is SEQ ID NO. 33, r2 is SEQ ID NO. 41, r4 is SEQ ID NO. 63, r5 is SEQ ID NO. 84 and r6 is SEQ ID NO. 90. In some embodiments, the single chain variable fragment CD3 binding protein comprises an amino acid sequence wherein r1 is SEQ ID NO. 30, r2 is SEQ ID NO. 44, r3 is SEQ ID NO. 58, r4 is SEQ ID NO. 66 and r5 is SEQ ID NO. 85. In some embodiments, the single chain variable fragment CD3 binding protein comprises an amino acid sequence wherein r1 is SEQ ID NO. 40, r2 is SEQ ID NO. 45, r3 is SEQ ID NO. 56, r5 is SEQ ID NO. 78 and r6 is SEQ ID NO. 93. In some embodiments, the single chain variable fragment CD3 binding protein comprises an amino acid sequence wherein r1 is SEQ ID NO. 36, r2 is SEQ ID NO. 50, r3 is SEQ ID NO. 59, r4 is SEQ ID NO. 72 and r5 is SEQ ID NO. 75.

In some embodiments, the single chain variable fragment CD3 binding protein has an amino acid sequence selected from SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 94, and SEQ ID NO. 95. In some embodiments, the single chain variable fragment CD3 binding protein has the amino acid sequence set forth as SEQ ID NO. 8. In some embodiments, the single chain variable fragment CD3 binding protein has the amino acid sequence set forth as SEQ ID NO. 9. In some embodiments, the single chain variable fragment CD3 binding protein has the amino acid sequence set forth as SEQ ID NO. 14. In some embodiments, the single chain variable fragment CD3 binding protein has the amino acid sequence set forth as SEQ ID NO. 19. In some embodiments, the single chain variable fragment CD3 binding protein has the amino acid sequence set forth as SEQ ID NO. 94. In some embodiments, the single chain variable fragment CD3 binding protein comprises an amino acid sequence comprising a linker, wherein said linker comprises the amino acid sequence as set forth in GGGGSGGGGSGGGGS (SEQ ID NO: 1). In some embodiments, the single chain variable fragment CD3 binding protein binds to CD3 selected from human CD3 and cynomolgus CD3. In some embodiments, the single chain variable fragment CD3 binding protein binds to human CD3 and cynomolgus CD3 with comparable binding affinity (Kd). In some embodiments, the single chain variable fragment CD3 binding protein binds to human CD3 with a human Kd (hKd) between about 1 nM and about 200 nM and to cynomolgus CD3 with a cynomolgus Kd (cKd) between about 1 nM and about 300 nM. In some embodiments, the hKd and the cKd are between about 3 nM to about 5 nM, about 6 nM to about 10 nM, about 11 nM to about 20 nM, about 25 nM to about 40 nM,about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM.

In some embodiments, the single chain variable fragment CD3 binding protein binds to human CD3 with a human Kd (hKd), binds to cynomolgus CD3 with a cynomolgus Kd (cKd), and the hKd and the cKd are about the same as the binding affinity towards CD3 of a protein which has the sequence as set forth in wt anti-CD3 (SEQ ID NO. 22). In some embodiments, the single chain variable fragment CD3 binding protein binds to human CD3 with a human Kd (hKd), binds to cynomolgus CD3 with a cynomolgus Kd (cKd), and the hKd and the cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein binds to human CD3 with a human Kd (hKd), binds to cynomolgus CD3 with a cynomolgus Kd (cKd), and the hKd and the cKd are between about 3-fold to about 5-fold higher than the binding affinity towards CD3 of a protein which has the sequence as set forth in wt anti-CD3 (SEQ ID NO.22). In some embodiments, the single chain variable fragment CD3 binding protein binds to human CD3 with a human Kd (hKd), binds to cynomolgus CD3 with a cynomolgus Kd (cKd), and the hKd and the cKd are between about 6-fold to about 15-fold higher than the binding affinity towards CD3 of a protein which has the sequence as set forth in wt anti-CD3 (SEQ ID NO.22). In some embodiments, the single chain variable fragment CD3 binding protein binds to human CD3 with a human Kd (hKd), binds to cynomolgus CD3 with a cynomolgus Kd (cKd), and the hKd and the cKd are between about 20-fold to about 50-fold higher than the binding affinity towards CD3 of a protein which has the sequence as set forth in wt anti-CD3 (SEQ ID NO.22).

In some embodiments, the single chain variable fragment CD3 binding protein has the amino acid sequence set forth as SEQ ID NO. 8, and the hKd and cKd are about the same as the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 8, and the hKd and the cKd are between about 3 nM and about 5 nM. In some embodiments, the single chain variable fragment CD3 binding protein has the amino acid sequence set forth as SEQ ID NO. 9, and the hKd and the cKd are about the same as the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 9, and the hKd and the cKd are between about 3 nM and about 5 nM. In some embodiments, the single chain variable fragment CD3 binding protein has the amino acid sequence set forth as SEQ ID NO. 10, and the hKd and cKd are about the same as the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 10, and the hKd and the cKd are between about 3 nM and about 5 nM. In some embodiments, the single chain variable fragment CD3 binding protein has the amino acid sequence set forth as SEQ ID NO. 11, and wherein the hKd and cKd are about about the same as the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 11, and the hKd and the cKd are between about 3 nM and about 5 nM. In some embodiments, the single chain variable fragment CD3 binding protein has the amino acid sequence set forth as SEQ ID NO. 12, and the hKd and cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 12, and the hKd and the cKd are between about 6 nM and about 10 nM. In some embodiments, the single chain variable fragment CD3 binding protein has the amino acid sequence set forth as SEQ ID NO. 13, and the hKd and the cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 13, and the hKd and the cKd are between about 6 nM and about 10 nM. In some embodiments, the single chain variable fragment CD3 binding protein has the amino acid sequence set forth as SEQ ID NO. 14, and the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 14, and the hKd and the cKd are between about 11 nM and 20 nM. In some embodiments, the single chain variable fragment CD3 binding protein has the amino acid sequence set forth as SEQ ID NO. 15, and the hKd and cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 15, and the hKd and the cKd are between about 25 nM and about 40 nM. In some embodiments, the single chain variable fragment CD3 binding protein has the amino acid sequence set forth as SEQ ID NO. 16, and the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 16, and the hKd iand the cKd are between about 11 nM and 20 nM. In some embodiments, the single chain variable fragment CD3 binding protein has the amino acid sequence set forth as SEQ ID NO. 17, and the hKd and cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 17, and the hKd and the cKd are between about 40 nM and about 60 nM. In some embodiments, the single chain variable fragment CD3 binding protein has the amino acid sequence set forth as SEQ ID NO. 18, and the hKd and cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 18, and the hKd and the cKd are between about 11 nM and 20 nM. In some embodiments, the single chain variable fragment CD3 binding protein has the amino acid sequence set forth as SEQ ID NO. 19, and the hKd and cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 19, and the hKd and the cKd are between about 40 nM and 60 nM. In some embodiments, the single chain variable fragment CD3 binding protein has the amino acid sequence set forth as SEQ ID NO. 20, and the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 20, and the hKd and the cKd are between about 11 nM and about 20 nM. In some embodiments, the single chain variable fragment CD3 binding protein has the amino acid sequence set forth as SEQ ID NO. 21, and the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 21, and the hKd and the cKd are between about 125 nM and about 140 nM. In some embodiments, the single chain variable fragment CD3 binding protein has the amino acid sequence set forth as SEQ ID NO. 94, and wherein the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 94, and the hKd and the cKd are between about 100 nM and about 120 nM. In some embodiments, the single chain variable fragment CD3 binding protein has the amino acid sequence set forth as SEQ ID NO. 95, and the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein has the amino acid sequence set forth as SEQ ID NO. 95, and the hKd and the cKd are between about 100 nM and about 120 nM.

Provided herein in another embodiment is a single chain variable fragment CD3 binding protein comprising the sequence set forth as SEQ ID NO. 22 (wt anti-CD3) wherein one or more amino acid residues selected from amino acid positions 27, 28, 29, 31, 33, 34, 54, 55, 57, 59, 61, 63, 65, 102, 105, 106, 108, 114, 163, 164, 165, 166, 168, 170, 171, 173, 193, 194, 195, 196, 197, 231, 235, 237, and 239 are substituted, wherein amino acid position 27 is substituted with asparagine, amino acid position 28 is substituted with glutamic acid, or methionine, amino acid position 29 is substituted with tyrosine, amino acid position 31 is substituted with asparagine, glycine, glutamic acid or threonine, amino acid position 33 is substituted with proline, amino acid position 34 is substituted with valine, leucine or isoleucine, amino acid position 54 is substituted with glycine, amino acid position 55 is substituted with serine, amino acid position 57 is substituted with lysine, amino acid position 59 is substituted with glutamic acid, amino acid position 61 is substituted with glutamic acid, amino acid position 63 is substituted with lysine, amino acid position 65 is substituted with aspartic acid, glutamic acid, alanine, or glutamine, amino acid position 102 is substituted with alanine or threonine, amino acid position 105 is substituted with asparagine, amino acid position 106 is substituted with aspartic acid, amino acid position 108 is substituted with histidine, proline, glutamine, glycine, or leucine, amino acid position 114 is substituted with threonine, amino acid position 163 is substituted with alanine, amino acid position 164 is substituted with glutamic acid, amino acid position 165 is substituted with tyrosine, amino acid position 166 is substituted with phenylalanine, lysine, or serine, amino acid position 168 is substituted with tyrosine, amino acid position 170 is substituted with valine, amino acid position 171 is substituted with aspartic acid, lysine, valine, or histidine, amino acid position 173 is substituted with tyrosine, amino acid position 193 is substituted with isoleucine, amino acid position 194 is substituted with glutamic acid, tyrosine, asparagine, or serine, amino acid position 195 is substituted with leucine, glutamic acid, isoleucine, methionine, or valine, amino acid position 196 is substituted with asparagine, or glycine, amino acid position 197 is substituted with valine, amino acid position 231 is substituted with threonine, or alanine, amino acid position 235 is substituted with aspartic acid, or alanine, amino acid position 237 is substituted with serine, and amino acid position 239 is substituted with alanine, or isoleucine. In some embodiments, the single chain variable fragment CD3 binding protein comprises one or more additional substitutions in amino acid positions other than positions 27, 28, 29, 31, 33, 34, 54, 55, 57, 59, 61, 63, 65, 102, 105, 106, 108, 114, 163, 164, 165, 166, 168, 170, 171, 173, 193, 194, 195, 196, 197, 231, 235,237, and 239. In some embodiments, the single chain variable fragment CD3 binding protein comprises a substitution in position 27. In some embodiments, the single chain variable fragment CD3 binding protein comprises a substitution in position 28. In some embodiments, the single chain variable fragment CD3 binding protein comprises a substitution in position 29. In some embodiments, the single chain variable fragment CD3 binding protein comprises a substitution in position 31. In some embodiments, the single chain variable fragment CD3 binding protein comprises a substitution in position 33. In some embodiments, the single chain variable fragment CD3 binding protein comprises a substitution in position 34. In some embodiments, the single chain variable fragment CD3 binding protein comprises a substitution in position 54. In some embodiments, the single chain variable fragment CD3 binding protein comprises a substitution in position 55. In some embodiments, the single chain variable fragment CD3 binding protein comprises a substitution in position 57. In some embodiments, the single chain variable fragment CD3 binding protein comprises a substitution in position 59. In some embodiments, the single chain variable fragment CD3 binding protein comprises a substitution in position 61. In some embodiments, the single chain variable fragment CD3 binding protein comprises a substitution in position 63. In some embodiments, the single chain variable fragment CD3 binding protein comprises a substitution in position 65. In some embodiments, the single chain variable fragment CD3 binding protein comprises a substitution in position 102. In some embodiments, the single chain variable fragment CD3 binding protein comprises a substitution in position 105. In some embodiments, the single chain variable fragment CD3 binding protein comprises a substitution in position 106. In some embodiments, the single chain variable fragment CD3 binding protein comprises a substitution in position 108. In some embodiments, the single chain variable fragment CD3 binding protein comprises a substitution in position 114. In some embodiments, the single chain variable fragment CD3 binding protein comprises a substitution in position 163. In some embodiments, the single chain variable fragment CD3 binding protein comprises a substitution in position 164. In some embodiments, the single chain variable fragment CD3 binding protein comprises a substitution in position 165. In some embodiments, the single chain variable fragment CD3 binding protein comprises a substitution in position 166. In some embodiments, the single chain variable fragment CD3 binding protein comprises a substitution in position 168. In some embodiments, the single chain variable fragment CD3 binding protein comprises a substitution in position 170. In some embodiments, the single chain variable fragment CD3 binding protein comprises a substitution in position 171. In some embodiments, the single chain variable fragment CD3 binding protein comprises a substitution in position 173. In some embodiments, the single chain variable fragment CD3 binding protein comprises a substitution in position 193. In some embodiments, the single chain variable fragment CD3 binding protein comprises a substitution in position 194. In some embodiments, the single chain variable fragment CD3 binding protein comprises a substitution in position 195. In some embodiments, the single chain variable fragment CD3 binding protein comprises a substitution in position 196. In some embodiments, the single chain variable fragment CD3 binding protein comprises a substitution in position 197. In some embodiments, the single chain variable fragment CD3 binding protein comprises a substitution in position 231. In some embodiments, the single chain variable fragment CD3 binding protein comprises a substitution in position 235. In some embodiments, the single chain variable fragment CD3 binding protein comprises a substitution in position 237. In some embodiments, the single chain variable fragment CD3 binding protein comprises a substitution in position 239. In some embodiments, the single chain variable fragment CD3 binding protein comprises substitutions in positions 34, 65, 102, 163, 197, and 231. In some embodiments, the single chain variable fragment CD3 binding protein comprises substitutions in positions 28, 57, 166, and 235. In some embodiments, the single chain variable fragment CD3 binding protein comprises substitutions in positions 108, 168, 194, and 239. In some embodiments, the single chain variable fragment CD3 binding protein comprises substitutions in positions 28, 55, 114, 166, 195, and 237. In some embodiments, the single chain variable fragment CD3 binding protein comprises substitutions in positions 31, 63, 108, 170, and 194. In some embodiments, the single chain variable fragment CD3 binding protein comprises substitutions in positions 29, 65, 108, 166, 195, and 231. In some embodiments, the single chain variable fragment CD3 binding protein comprises substitutions in positions 27, 59, 102, 173, and 194. In some embodiments, the single chain variable fragment CD3 binding protein comprises substitutions in positions 31, 65, 108, 171, and 193. In some embodiments, the single chain variable fragment CD3 binding protein comprises substitutions in positions 34, 164, and 195. In some embodiments, the single chain variable fragment CD3 binding protein comprises substitutions in positions 33, 105, 171, 195, and 231. In some embodiments, the single chain variable fragment CD3 binding protein comprises substitutions in positions 31, 65, 108, 170, and 194. In some embodiments, the single chain variable fragment CD3 binding protein comprises substitutions in positions 27, 106, 171, and 195. In some embodiments, the single chain variable fragment CD3 binding protein comprises substitutions in positions 31, 54, 165, 196, and 235. In some embodiments, the single chain variable fragment CD3 binding protein comprises substitutions in positions 28, 59, 108, 166, and 196. In some embodiments, the single chain variable fragment CD3 binding protein comprises substitutions in positions 34, 61, 108, 194, and 239. In some embodiments, the single chain variable fragment CD3 binding protein comprises substitutions in positions 31, 65, 108, 171, and 194.

In some embodiments, the single chain variable fragment CD3 binding protein comprises a sequence wherein amino acid positions 34, 65, 102, 163, 197, and 231 are substituted, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 34, 65, 102, 163, 197, and 231 are substituted, and wherein the hKd and the cKd are between about 3 nM and about 5 nM. In some embodiments, the single chain variable fragment CD3 binding protein comprises a sequence wherein amino acid positions 28, 57, 166, and 235 are substituted, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 28, 57, 166, and 235 are substituted, and wherein the hKd and the cKd are between about 3 nM and about 5 nM.

In some embodiments, the single chain variable fragment CD3 binding protein comprises a sequence wherein amino acid positions 108, 168, 194, and 239 are substituted, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 108, 168, 194, and 239 are substituted, and wherein the hKd and the cKd are between about 3 nM and about 5 nM. In some embodiments, the single chain variable fragment CD3 binding protein comprises a sequence wherein amino acid positions 28, 55, 114, 166, 195, and 237 are substituted, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 28, 55, 114, 166, 195, and 237 are substituted, and wherein the hKd and the cKd are between about 3 nM and about 5 nM. In some embodiments, the single chain variable fragment CD3 binding protein comprises a sequence wherein amino acid positions 31, 63, 108, 170, and 194 are substituted, and wherein the hKd and the cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 31, 63, 108, 170, and 194 are substituted, and wherein the hKd and the cKd are between about 6 nM and about 10 nM. In some embodiments, the single chain variable fragment CD3 binding protein comprises a sequence wherein amino acid positions 29, 65, 108, 166, 195, and 231 are substituted, and wherein the hKd and the cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 29, 65, 108, 166, 195, and 231 are substituted, and wherein the hKd and the cKd are between about 6 nM and about 10 nM. In some embodiments, the single chain variable fragment CD3 binding protein comprises a sequence wherein amino acid positions 27, 59, 102, 173, and 194 are substituted, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 27, 59, 102, 173, and 194 are substituted, and wherein the hKd and the cKd are between about 11 nM and 20 nM. In some embodiments, the single chain variable fragment CD3 binding protein comprises a sequence wherein amino acid positions 31, 65, 108, 171, and 193 are substituted, and wherein the hKd and the cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 31, 65, 108, 171, and 193 are substituted, and wherein the hKd and the cKd are between about 25 nM and about 40 nM. In some embodiments, the single chain variable fragment CD3 binding protein comprises a sequence wherein amino acid positions 34, 164, and 195 are substituted, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 34, 164, and 195 are substituted, and wherein the hKd and the cKd are between about 11 nM and 20 nM. In some embodiments, the single chain variable fragment CD3 binding protein comprises a sequence wherein amino acid positions 33, 105, 171, 195, and 231 are substituted, and wherein the hKd and the cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 33, 105, 171, 195, and 231 are substituted, and wherein the hKd and the cKd are between about 40 nM and about 60 nM. In some embodiments, the single chain variable fragment CD3 binding protein comprises a sequence wherein amino acid positions 31, 65, 108, 170, and 194 are substituted, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 31, 65, 108, 170, and 194 are substituted, and wherein the hKd and the cKd are between about 11 nM and 20 nM. In some embodiments, the single chain variable fragment CD3 binding protein comprises a sequence wherein amino acid positions 27, 106, 171, and 195 are substituted, and wherein the hKd and the cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 27, 106, 171, and 195 are substituted, and wherein the hKd and the cKd are between about 40 nM and 60 nM. In some embodiments, the single chain variable fragment CD3 binding protein comprises a sequence wherein amino acid positions 31, 54, 165, 196, and 235 are substituted, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 31, 54, 165, 196, and 235 are substituted (10B2), and wherein the hKd and the cKd are between about 11 nM and about 20 nM. In some embodiments, the single chain variable fragment CD3 binding protein comprises a sequence wherein amino acid positions 28, 59, 108, 166, and 196 are substituted, and wherein the hKd and the cKd are about 25-fold to about 50-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 28, 59, 108, 166, and 196 are substituted, and wherein the hKd and the cKd are between about 125 nM and about 140 nM. In some embodiments, the single chain variable fragment CD3 binding protein comprises a sequence wherein amino acid positions 34, 61, 108, 194, and 239 are substituted, and wherein the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 34, 61, 108, 194, and 239 are substituted, and wherein the hKd and the cKd are between about 100 nM and about 120 nM. In some embodiments, the single chain variable fragment CD3 binding protein comprises a sequence wherein amino acid positions 31, 65, 108, 171, and 194 are substituted, and wherein the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid positions 31, 65, 108, 171, and 194 are substituted, and wherein the hKd and the cKd are between about 100 nM and about 120 nM.

Provided herein in a further embodiment is a single chain variable fragment CD3 binding protein comprising an amino acid sequence as set forth in wt anti-CD3 (SEQ ID NO: 22), comprising a variable heavy chain region (VH), a variable light chain region (VL), a linker comprising the amino acid sequence as set forth in GGGGSGGGGSGGGGS (SEQ ID NO: 1), wherein VH comprises complementarity determining regions CDR1, CDR2, and CDR3, wherein VL comprises complementarity determining regions LC CDR1, LC CDR2, and LC CDR3, comprising at least one mutation in CDR1, CDR2 or CDR3 of VH, and LC CDR1, LC CDR2 or LC CDR3 of VL, wherein the at least one mutation is not in amino acid positions 26, 30, 32, 35, 50, 51, 52, 53, 56, 58, 60, 62, 64, 66, 67, 101, 103, 104, 107, 109, 110, 111, 112, 113, 167, 169, 172, 174, 175, 176, 192, 198, 232, 233, 234, 236, or 238. In some embodiments, the single chain variable fragment CD3 binding protein comprises at least one mutation in amino acid position selected from 27, 28, 29, 31, 33, 34, 54, 55, 57, 59, 61, 63, 65, 102, 105, 106, 108, 114, 163, 164, 165, 166, 168, 170, 171, 173, 193, 194, 195, 196, 197, 231, 235, 237, and 239. In some embodiments, amino acid position 34 is mutated to isoleucine, position 65 is mutated to glutamine, position 102 is mutated to alanine, position 163 is mutated to alanine, position 197 is mutated to valine, and position 231 is mutated to threonine. In some embodiments, amino acid position 28 is mutated to glutamic acid, position 57 is mutated to lysine, position 166 is mutated to phenylalanine, and position 235 is mutated to aspartic acid. In some embodiments, amino acid position 108 is mutated to histidine, position 168 is mutated to tyrosine, position 194 is mutated to serine, and position 239 is mutated to isoleucine. In some embodiments, amino acid position 28 is mutated to methionine, position 55 is mutated to serine, position 114 is mutated to threonine, position 166 is mutated to phenylalanine, position 195 is mutated to leucine, and position 237 is mutated to serine. In some embodiments, amino acid position 31 is mutated to threonine, position 63 is mutated to lysine, position 108 is mutated to proline, position 170 is mutated to valine, and position 194 is mutated to glutamic acid. In some embodiments, amino acid position 29 is mutated to tyrosine, position 65 is mutated to glutamic acid, position 108 is mutated to proline, position 166 is mutated to lysine, position 195 is mutated to glutamic acid, and position 231 is mutated to threonine. In some embodiments, amino acid position 27 is mutated to asparagine, position 59 is mutated to glutamic acid, position 102 is mutated to threonine, position 173 is mutated to tyrosine, and position 194 is mutated to tyrosine. In some embodiments, amino acid position amino acid position 31 is mutated to asparagine, position 65 is mutated to alanine, position 108 is mutated to glutamine, position 171 is mutated to aspartic acid, and position 193 is mutated to isoleucine. In some embodiments, amino acid position 34 is mutated to valine, position 164 is mutated to glutamic acid, and position 195 is mutated to isoleucine. In some embodiments, amino acid position 33 is mutated to proline, position 105 is mutated to asparagine, position 171 is mutated to lysine, position 195 is mutated to methionine, and position 231 is mutated to alanine. In some embodiments, amino acid position 31 is mutated to glycine, position 65 is mutated to glutamic acid, position 108 is mutated to proline, position 170 is mutated to valine, and position 194 is mutated to glutamic acid. In some embodiments, amino acid position 27 is mutated to asparagine, position 106 is mutated to aspartic acid, position 171 is mutated to histidine, and position 195 is mutated to valine. In some embodiments, amino acid position 31 is mutated to asparagine, position 54 is mutated to glycine, position 165 is mutated to tyrosine, position 196 is mutated to asparagine, and position 235 is mutated to alanine. In some embodiments, amino acid position 28 is mutated to glutamic acid, position 59 is mutated to glutamic acid, position 108 is mutated to leucine, position 166 is mutated to serine, and position 196 is mutated to glycine. In some embodiments, amino acid position 34 is substituted with leucine, amino acid position 61 is substituted with glutamic acid, amino acid position 108 is substituted with proline, amino acid position 194 is substituted with asparagine, and amino acid position 239 is substituted with alanine. In some embodiments, amino acid position 31 is substituted with glutamic acid, amino acid position 65 is substituted with aspartic acid, amino acid position 108 is substituted with glycine, amino acid position 171 is substituted with valine, and amino acid position 194 is substituted with glutamic acid.

In some embodiments, the single chain variable fragment CD3 binding protein comprises a sequence wherein amino acid position 34 is mutated to isoleucine, position 65 is mutated to glutamine, position 102 is mutated to alanine, position 163 is mutated to alanine, position 197 is mutated to valine, and position 231 is mutated to threonine, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid position 34 is mutated to isoleucine, position 65 is mutated to glutamine, position 102 is mutated to alanine, position 163 is mutated to alanine, position 197 is mutated to valine, and position 231 is mutated to threonine, wherein the hKd and the cKd are between about 3 nM and 5 nM. In some embodiments, the single chain variable fragment CD3 binding protein comprises a sequence wherein amino acid position 28 is mutated to glutamic acid, position 57 is mutated to lysine, position 166 is mutated to phenylalanine, and position 235 is mutated to aspartic acid, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid position 28 is mutated to glutamic acid, position 57 is mutated to lysine, position 166 is mutated to phenylalanine, and position 235 is mutated to aspartic acid, wherein the hKd and the cKd are between about 3 nM and about 5 nM. In some embodiments, the single chain variable fragment CD3 binding protein comprises a sequence wherein amino acid position 108 is mutated to histidine, position 168 is mutated to tyrosine, position 194 is mutated to serine, and position 239 is mutated to isoleucine, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid position 108 is mutated to histidine, position 168 is mutated to tyrosine, position 194 is mutated to serine, and position 239 is mutated to isoleucine, wherein the hKd and the cKd are between about 3 nM and about 5 nM. In some embodiments, the single chain variable fragment CD3 binding protein comprises a sequence wherein amino acid position 28 is mutated to methionine, position 55 is mutated to serine, position 114 is mutated to threonine, position 166 is mutated to phenylalanine, position 195 is mutated to leucine, and position 237 is mutated to serine, and wherein the hKd and the cKd are about the same as the binding affinity towards CD3 of a protein which has the sequence as set forth in wt anti-CD3 (SEQ ID NO. 22). In some embodiments, the single chain variable fragment CD3 binding protein comprises a sequence wherein amino acid position 28 is mutated to methionine, position 55 is mutated to serine, position 114 is mutated to threonine, position 166 is mutated to phenylalanine, position 195 is mutated to leucine, and position 237 is mutated to serine, wherein the hKd and the cKd are between about 3 nM and about 5 nM. In some embodiments, the single chain variable fragment CD3 binding protein comprises a sequence wherein amino acid position 31 is mutated to threonine, position 63 is mutated to lysine, position 108 is mutated to proline, position 170 is mutated to valine, and position 194 is mutated to glutamic acid, and wherein the hKd and the cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 31 is mutated to threonine, position 63 is mutated to lysine, position 108 is mutated to proline, position 170 is mutated to valine, and position 194 is mutated to glutamic acid, wherein the hKd and the cKd are between about 6 nM and about 10 nM. In some embodiments, the single chain variable fragment CD3 binding protein comprises a sequence wherein amino acid position 29 is mutated to tyrosine, position 65 is mutated to glutamic acid, position 108 is mutated to proline, position 166 is mutated to lysine, position 195 is mutated to glutamic acid, and position 231 is mutated to threonine, and wherein the hKd and the cKd are between about 1.5-fold to about 2-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 29 is mutated to tyrosine, position 65 is mutated to glutamic acid, position 108 is mutated to proline, position 166 is mutated to lysine, position 195 is mutated to glutamic acid, and position 231 is mutated to threonine, wherein the hKd and the cKd are between about 6 nM and about 10 nM. In some embodiments, the single chain variable fragment CD3 binding protein comprises a sequence wherein amino acid position 27 is mutated to asparagine, position 59 is mutated to glutamic acid, position 102 is mutated to threonine, position 173 is mutated to tyrosine, and position 194 is mutated to tyrosine, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 27 is mutated to asparagine, position 59 is mutated to glutamic acid, position 102 is mutated to threonine, position 173 is mutated to tyrosine, and position 194 is mutated to tyrosine, wherein the hKd and the cKd are between about 11 nM and 20 nM. In some embodiments, the single chain variable fragment CD3 binding protein comprises a sequence wherein amino acid position 31 is mutated to asparagine, position 65 is mutated to alanine, position 108 is mutated to glutamine, position 171 is mutated to aspartic acid, and position 193 is mutated to isoleucine, and wherein the hKd and the cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 31 is mutated to asparagine, position 65 is mutated to alanine, position 108 is mutated to glutamine, position 171 is mutated to aspartic acid, and position 193 is mutated to isoleucine, wherein the hKd and the cKd are between about 25 nM and about 40 nM. In some embodiments, the single chain variable fragment CD3 binding protein comprises a sequence wherein amino acid position 34 is mutated to valine, position 164 is mutated to glutamic acid, and position 195 is mutated to isoleucine, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 34 is mutated to valine, position 164 is mutated to glutamic acid, and position 195 is mutated to isoleucine, wherein the hKd and the cKd are between about 11 nM and 20 nM. In some embodiments, the single chain variable fragment CD3 binding protein comprises a sequence wherein amino acid position 33 is mutated to proline, position 90 is mutated to asparagine, position 105 is mutated to asparagine, position 171 is mutated to lysine, position 195 is mutated to methionine, and position 231 is mutated to alanine, and wherein the hKd and the cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 33 is mutated to proline, position 90 is mutated to asparagine, position 105 is mutated to asparagine, position 171 is mutated to lysine, position 195 is mutated to methionine, and position 231 is mutated to alanine, wherein the hKd and the cKd are between about 40 nM and 60 nM. In some embodiments, the single chain variable fragment CD3 binding protein comprises a sequence wherein amino acid position 31 is mutated to glycine, position 65 is mutated to glutamic acid, position 108 is mutated to proline, position 170 is mutated to valine, and position 194 is mutated to glutamic acid, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 31 is mutated to glycine, position 65 is mutated to glutamic acid, position 108 is mutated to proline, position 170 is mutated to valine, and position 194 is mutated to glutamic acid, wherein the hKd and the cKd are between about 11 nM and 20 nM. In some embodiments, the single chain variable fragment CD3 binding protein comprises a sequence wherein amino acid position 27 is mutated to asparagine, position 106 is mutated to aspartic acid, position 171 is mutated to histidine, and position 195 is mutated to valine, and wherein the hKd and the cKd are about 6-fold to about 15-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 27 is mutated to asparagine, position 106 is mutated to aspartic acid, position 171 is mutated to histidine, and position 195 is mutated to valine, wherein the hKd and the cKd are between about 40 nM and 60 nM. In some embodiments, the single chain variable fragment CD3 binding protein comprises a sequence wherein amino acid position 31 is mutated to asparagine, position 54 is mutated to glycine, position 165 is mutated to tyrosine, position 196 is mutated to asparagine, and position 235 is mutated to alanine, and wherein the hKd and the cKd are about 3-fold to about 5-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 31 is mutated to asparagine, position 54 is mutated to glycine, position 165 is mutated to tyrosine, position 196 is mutated to asparagine, and position 235 is mutated to alanine, wherein the hKd and the cKd are between about 11 nM and about 20 nM. In some embodiments, the single chain variable fragment CD3 binding protein comprises a sequence wherein amino acid position 28 is mutated to glutamic acid, position 59 is mutated to glutamic acid, position 108 is mutated to leucine, position 166 is mutated to serine, and position 196 is mutated to glycine, and wherein the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 28 is mutated to glutamic acid, position 59 is mutated to glutamic acid, position 108 is mutated to leucine, position 166 is mutated to serine, and position 196 is mutated to glycine, wherein the hKd and the cKd are between about 125 nM and about 140 nM. In some embodiments, the single chain variable fragment CD3 binding protein comprises a sequence wherein amino acid position 34 is mutated to leucine, amino acid position 61 is mutated to glutamic acid, amino acid position 108 is mutated to proline, amino acid position 194 is mutated to asparagine, amino acid position 239 is mutated to alanine, and wherein the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 34 is mutated to leucine, amino acid position 61 is mutated to glutamic acid, amino acid position 108 is mutated to proline, amino acid position 194 is mutated to asparagine, amino acid position 239 is mutated to alanine, wherein the hKd and the cKd are between about 100 nM and about 120 nM. In some embodiments, the single chain variable fragment CD3 binding protein comprises a sequence wherein amino acid position 31 is mutated to glutamic acid, amino acid position 65 is mutated to aspartic acid, amino acid position 108 is mutated to glycine, amino acid position 171 is mutated to valine, and amino acid position 194 is mutated to glutamic acid, and wherein the hKd and the cKd are about 20-fold to about 50-fold higher than the binding affinity towards CD3 of a protein which has the sequence as set forth in wt anti-CD3 (SEQ ID NO. 22). In some embodiments, the single chain variable fragment CD3 binding protein comprises a sequence wherein amino acid position 31 is mutated to glutamic acid, amino acid position 65 is mutated to aspartic acid, amino acid position 108 is mutated to glycine, amino acid position 171 is mutated to valine, and amino acid position 194 is mutated to glutamic acid, and wherein the hKd and the cKd are between about 100 nM and about 120 nM.

In some embodiments, the single chain variable fragment CD3 binding protein does not bind to mouse CD3.

In one embodiment is provided a polynucleotide encoding a single chain variable fragment CD3 binding protein according to the present disclosure. In one embodiment is provided a vector comprising the polynucleotide described herein. In one embodiment is provided a host cell transformed with the vector described herein. In a further embodiment is provided a pharmaceutical composition comprising (i) a single chain variable fragment CD3 binding protein according to the present disclosure, the polynucleotide according to present disclosure, the vector according to present disclosure or the host cell according to present disclosure, and (ii) a pharmaceutically acceptable carrier.

In another embodiment is provided a process for the production of a single chain variable fragment CD3 binding protein according to the present disclosure, said process comprising culturing a host transformed or transfected with a vector comprising a nucleic acid sequence encoding single chain variable fragment CD3 binding protein according to the present disclosure under conditions allowing the expression of the single chain variable fragment CD3 binding protein and recovering and purifying the produced protein from the culture.

In another embodiment is provided a method for the treatment or amelioration of a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease comprising the administration of the single chain variable fragment CD3 binding protein according to the present disclosure, to a subject in need thereof. In some embodiments, the subject is human. In some embodiments, the method further comprises administration of an agent in combination with the single chain variable fragment CD3 binding protein according to the present disclosure.

Provided herein in one embodiment is a multispecific binding protein comprising the single chain variable fragment CD3 binding protein according the present disclosure. One embodiment describes an antibody comprising the single chain variable fragment CD3 binding protein according to the present disclosure. A further embodiment provides a multispecific antibody, a bispecific antibody, a single domain antibody, a variable heavy domain, a peptide, or a ligand, comprising the single chain variable fragment CD3 binding protein according to the present disclosure. Described herein in one embodiment is an antibody comprising the single chain variable fragment CD3 binding protein according to the present disclosure, wherein said antibody is a scFv antibody. Another embodiment describes a multispecific binding protein or antibody comprising the single chain variable fragment CD3 binding protein according to the present disclosure and a serum albumin binding domain.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

### BRIEF DESCRIPTION OF THE DRAWINGS

FIG. 1 illustrates phage titration on biotin-CD3ε and biotin-HSA.

FIG. 2 illustrates the amino acid sequence of the human CD3 binding protein (SEQ ID NO: 22). HC CDR1 is indicated in the first shaded sequence (GFTFNKYAMN (SEQ ID NO: 23)), HC CDR2 is indicated in the second shaded sequence (RIRSKYNNYATYYADSVK (SEQ ID NO: 24)), HC CDR3 is indicated in the third shaded sequence (HGNFGNSYISYWAY (SEQ ID NO: 25)), LC CDR1is indicated in the fourth shaded sequence (GSSTGAVTSGNYPN (SEQ ID NO: 26)), LC CDR2 is indicated in the fifth shaded sequence (GTKFLAP (SEQ ID NO: 27)), and CDR3 is indicated in the sixth shaded sequence (VLWYSNRWV (SEQ ID NO: 28)).

FIG. 3 illustrates the profiles of the sixteen clones selected for more precise K_{d} determinations.

FIG. 4 illustrates the temperature of hydrophobic exposure (Tₕ ° C.) for several anti-huCD3ε scFv variants.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e. after drug absorption and distribution are complete). The half-time for drug elimination can be accurately determined from such a graph.

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The term “Framework” or “FR” residues (or regions) refer to variable domain residues other than the CDR or hypervariable region residues as herein defined. A “human consensus framework” is a framework which represents the most commonly occurring amino acid residue in a selection of human immunoglobulin VL or VH framework sequences.

As used herein, “Variable region” or “variable domain” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a (3-sheet configuration, connected by three CDRs, which form loops connecting, and in some cases forming part of, the (3sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity. “Variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. It is not intended that CDRs of the present disclosure necessarily correspond to the Kabat numbering convention.

As used herein, the term “binding affinity” refers to the affinity of the proteins described in the disclosure to their binding targets, and is expressed numerically using “Kd” values. If two or more proteins are indicated to have comparable binding affinities towards their binding targets, then the Kd values for binding of the respective proteins towards their binding targets, are within ±2-fold of each other. If two or more proteins are indicated to have comparable binding affinities towards single binding target, then the Kd values for binding of the respective proteins towards said single binding target, are within ±2-fold of each other. If a protein is indicated to bind two or more targets with comparable binding affinities, then the Kd values for binding of said protein to the two or more targets are within ±2-fold of each other. In general, a higher Kd value corresponds to a weaker binding. In some embodiments, the “Kd” is measured by a radiolabeled antigen binding assay (MA) or surface plasmon resonance assays using a BIAcore™-2000 or a BIAcore™-3000 (BIAcore, Inc., Piscataway, N.J.). In certain embodiments, an “on-rate” or “rate of association” or “association rate” or “kon” and an “off-rate” or “rate of dissociation” or “dissociation rate” or “koff” are also determined with the surface plasmon resonance technique using a BIAcore™-2000 or a BIAcore™-3000 (BIAcore, Inc., Piscataway, N.J.). In additional embodiments, the “Kd”, “kon”, and “koff” are measured using the Octet® Systems (Pall Life Sciences).

Described herein are single chain variable fragment CD3 binding proteins, pharmaceutical compositions as well as nucleic acids, recombinant expression vectors, and host cells for making such single chain variable fragment CD3 binding proteins. Also provided are methods of using the disclosed single chain variable fragment CD3 binding proteins in the prevention, and/or treatment of diseases, conditions and disorders. In some embodiments, the single chain variable fragment CD3 binding proteins are capable of specifically binding to a CD 3 domain, as well as a target antigen and a half-life extension domain, such as a single domain binding antibody to human serum albumin (HSA).

### CD3 Binding Domain

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the T cell receptor (TCR) as well as and CD3 ζ (zeta) altogether to comprise the T cell receptor complex. Clustering of CD3 on T cells, such as by immobilized anti-CD3 antibodies leads to T cell activation similar to the engagement of the T cell receptor but independent of its clone-typical specificity.

In one aspect, the single chain variable fragment CD3 binding proteins described herein comprise a domain which specifically binds to CD3. In one aspect, the single chain variable fragment CD3 binding proteins described herein comprise a domain which specifically binds to human CD3. In one aspect, the single chain variable fragment CD3 binding proteins described herein comprise a domain which specifically binds to cynomolgus CD3. In one aspect, the single chain variable fragment CD3 binding proteins described herein comprise a domain which binds to human CD3 and cynomolgus CD3. In some embodiments, the single chain variable fragment CD3 binding proteins described herein comprise a domain which specifically binds to CD3y. In some embodiments, the single chain variable fragment CD3 binding proteins described herein comprise a domain which specifically binds to CD3δ. In some embodiments, the single chain variable fragment CD3 binding proteins described herein comprise a domain which specifically binds to CD3ε.

In another aspect is provided a multispecific binding protein comprising a single chain variable fragment CD3 binding protein according to the present disclosure. In some embodiments, the multispecific protein comprising a single chain variable fragment CD3 binding protein according to the present disclosure specifically binds to the T cell receptor (TCR). In certain instances, the multispecific protein comprising a single chain variable fragment CD3 binding protein according to the present disclosure binds the a chain of the TCR. In certain instances, multispecific protein comprising a single chain variable fragment CD3 binding protein according to the present disclosure binds the R chain of the TCR.

In certain embodiments, the CD3 binding domain of the single chain variable fragment CD3 binding proteins described herein exhibit not only potent CD3 binding affinities with human CD3, but show also excellent crossreactivity with the respective cynomolgus monkey CD3 proteins. In some instances, the CD3 binding domain of the single chain variable fragment CD3 binding proteins are cross-reactive with CD3 from cynomolgus monkey. In certain instances, the Kd for binding human CD3 (hKd) is about the same as the Kd for binding cynomolgus CD3 (cKd). In certain instances, the ratio between hKd and cKd (hKd:cKd) is between about 20:1 to about 1:2.

In some embodiments, the CD3 binding domain of the single chain variable fragment CD3 binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the single chain variable fragment CD3 binding protein will ultimately be used. For example, for use in humans, it may be beneficial for the CD3 binding domain of the single chain variable fragment CD3 binding protein to comprise human or humanized residues from the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (CDR2), and heavy chain complementary determining region 3 (CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In certain instances, the complementary determining regions of the heavy chain and/or the light chain are derived from known anti-CD3 antibodies, such as, for example, muromonab-CD3 (OKT3), otelixizumab (TRX4), teplizumab (MGA031), visilizumab (Nuvion), SP34, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In one embodiment, the anti-CD3 binding domain is a single chain variable fragment (scFv) comprising a light chain and a heavy chain of an amino acid sequence provided herein. As used herein, “single chain variable fragment” or “scFv” refers to an antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived. In an embodiment, the anti-CD3 binding domain comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region- scFv linker-heavy chain variable region or heavy chain variable region- scFv linker-light chain variable region.

In some instances, scFvs which bind to CD3 are prepared according to known methods. For example, scFv molecules can be produced by linking VH and VL regions together using flexible polypeptide linkers. The scFv molecules comprise a scFv linker (e.g., a Ser-Gly linker) with an optimized length and/or amino acid composition. Accordingly, in some embodiments, the length of the scFv linker is such that the VH or VL domain can associate intermolecularly with the other variable domain to form the CD3 binding site. In certain embodiments, such scFv linkers are “short”, i.e. consist of 0, 1, 2, 3, 4, 5, 6, 7, 8, 9, 10, 11 or 12 amino acid residues. Thus, in certain instances, the scFv linkers consist of about 12 or less amino acid residues. In the case of 0 amino acid residues, the scFv linker is a peptide bond. In some embodiments, these scFv linkers consist of about 3 to about 15, for example 8, 10 or 15 contiguous amino acid residues. Regarding the amino acid composition of the scFv linkers, peptides are selected that confer flexibility, do not interfere with the variable domains as well as allow inter-chain folding to bring the two variable domains together to form a functional CD3 binding site. For example, scFv linkers comprising glycine and serine residues generally provide protease resistance. In some embodiments, linkers in a scFv comprise glycine and serine residues. The amino acid sequence of the scFv linkers can be optimized, for example, by phage-display methods to improve the CD3 binding and production yield of the scFv. Examples of peptide scFv linkers suitable for linking a variable light chain domain and a variable heavy chain domain in a scFv include but are not limited to (GS)ₙ (SEQ ID NO: 96), (GGS)ₙ (SEQ ID NO: 97), (GGGS)ₙ (SEQ ID NO: 98), (GGSG)ₙ (SEQ ID NO: 99), (GGSGG)ₙ (SEQ ID NO: 100), (GGGGS)ₙ (SEQ ID NO: 101), (GGGGG)ₙ (SEQ ID NO: 102), or (GGG)ₙ (SEQ ID NO: 103), wherein n is 1, 2, 3, 4, 5, 6, 7, 8, 9, or 10. In one embodiment, the scFv linker can be (GGGGS)₄ (SEQ ID NO: 104) or (GGGGS)₃ (SEQ ID NO: 1). Variation in the linker length may retain or enhance activity, giving rise to superior efficacy in activity studies.

In some embodiments, CD3 binding domain of a single chain variable fragment CD3 binding protein has an affinity to CD3 on CD3 expressing cells with a Kd of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In some embodiments, the CD3 binding domain of a single chain variable fragment CD3 binding protein has an affinity to CD3ε, γ, or δ with a K_{d} of 1000 nM or less, 500 nM or less, 200 nM or less, 100 nM or less, 80 nM or less, 50 nM or less, 20 nM or less, 10 nM or less, 5 nM or less, 1 nM or less, or 0.5 nM or less. In further embodiments, CD3 binding domain of a single chain variable fragment CD3 binding protein has low affinity to CD3, i.e., about 100 nM or greater.

In certain embodiments, the single chain variable fragment CD3 binding proteins described herein bind to human CD3 with a human Kd (hKd) and to cynomolgus CD3 with a cyno Kd (cKd). In some embodiments, hKd and cKd are between about between about 1 nM to about 2 nM, about 3 nM to about 5 nM, about 6 nM to about 10 nM, about 11 nM to about 20 nM, about 25 nM to about 40 nM,about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM.

In some embodiments, the hKd and cKd of the single chain variable fragment CD3 binding proteins is about the same as the Kd of a CD3 binding protein having the sequence as set forth is SEQ ID NO. 22. In some embodiments, the hKd and cKd of the single chain variable fragment CD3 binding proteins is about 1.1 fold to about 1.5 fold the Kd of a CD3 binding protein having the sequence as set forth is SEQ ID NO. 22. In some embodiments, the hKd and cKd of the single chain variable fragment CD3 binding proteins is about 1.5 fold to about 2 fold the Kd of a CD3 binding protein having the sequence as set forth is SEQ ID NO. 22. In some embodiments, the hKd and cKd of the single chain variable fragment CD3 binding proteins is about 2.5 fold to about 3 fold the Kd of a CD3 binding protein having the sequence as set forth is SEQ ID NO. 22. In some embodiments, the hKd and cKd of the single chain variable fragment CD3 binding proteins is about 3 fold to about 5 fold the Kd of a CD3 binding protein having the sequence as set forth is SEQ ID NO. 22. In some embodiments, the hKd and cKd of the single chain variable fragment CD3 binding proteins is about 6 fold to about 15 fold the Kd of a CD3 binding protein having the sequence as set forth is SEQ ID NO. 22. In some embodiments, the hKd and cKd of the single chain variable fragment CD3 binding proteins is about 15 fold to about 20 fold the Kd of a CD3 binding protein having the sequence as set forth is SEQ ID NO. 22. In some embodiments, the hKd and cKd of the single chain variable fragment CD3 binding proteins is about 20 fold to about 50 fold the Kd of a CD3 binding protein having the sequence as set forth is SEQ ID NO. 22. In some embodiments, the hKd and cKd of the single chain variable fragment CD3 binding proteins is about 55 fold to about 70 fold the Kd of a CD3 binding protein having the sequence as set forth is SEQ ID NO. 22. In some embodiments, the hKd and cKd of the single chain variable fragment CD3 binding proteins is about 75 fold to about 100 fold the Kd of a CD3 binding protein having the sequence as set forth is SEQ ID NO. 22. In some embodiments, the hKd and cKd of the single chain variable fragment CD3 binding proteins is about 120 fold to about 200 fold the Kd of a CD3 binding protein having the sequence as set forth is SEQ ID NO. 22.

In some embodiments, the ratio between the hKd and cKd (hKd:cKd) ranges from about 20:1 to about 1:2. The affinity to bind to CD3 can be determined, for example, by the ability of the single chain variable fragment CD3 binding protein itself or its CD3 binding domain to bind to CD3 coated on an assay plate; displayed on a microbial cell surface; in solution; etc. The binding activity of the single chain variable fragment CD3 binding protein itself or its CD3 binding domain of the present disclosure to CD3 can be assayed by immobilizing the ligand (e.g., CD3) or the single chain variable fragment CD3 binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high or low pH, and the like and analyzed, for example, by Surface Plasmon Resonance (SPR).

In some embodiments, the single chain variable fragment CD3 binding protein has an amino acid sequence selected from SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 94), and SEQ ID NO. 95.

In some embodiments, the single chain variable fragment CD3 binding protein has an amino acid sequence set forth as SEQ ID NO. 8, wherein the hKd is about 3.8 nM, and wherein the cKd is about 3.5 nM. In some embodiments, the single chain variable fragment CD3 binding protein has an amino acid sequence set forth as SEQ ID NO. 9, wherein the hKd is about 4.1 nM, and wherein the cKd is about 3.4 nM. In some embodiments, the single chain variable fragment CD3 binding protein has an amino acid sequence set forth as SEQ ID NO. 10, wherein the hKd is about 4.3 nM, and wherein the cKd is about 4.2 nM. In some embodiments, the single chain variable fragment CD3 binding protein has an amino acid sequence set forth as SEQ ID NO. 11, wherein the hKd is about 4.7 nM, and wherein the cKd is about 4.9 nM. In some embodiments, the single chain variable fragment CD3 binding protein has an amino acid sequence set forth as SEQ ID NO. 12, wherein the hKd is about 6.4 nM, and wherein the cKd is about 6.6 nM. In some embodiments, the single chain variable fragment CD3 binding protein has an amino acid sequence set forth as SEQ ID NO. 13, wherein the hKd is about 8 nM, and wherein the cKd is about 6.6 nM. In some embodiments, the single chain variable fragment CD3 binding protein has an amino acid sequence set forth as SEQ ID NO. 14, wherein the hKd is about 20 nM, and wherein the cKd is about 17 nM. In some embodiments, the single chain variable fragment CD3 binding protein has an amino acid sequence set forth as SEQ ID NO. 15, wherein the hKd is about 37 nM, and wherein the cKd is about 30 nM. In some embodiments, the single chain variable fragment CD3 binding protein has an amino acid sequence set forth as SEQ ID NO. 16, wherein the hKd is about 14 nM, and wherein the cKd is about 13 nM. In some embodiments, the single chain variable fragment CD3 binding protein has an amino acid sequence set forth as SEQ ID NO. 17, wherein the hKd is about 50 nM, and wherein the cKd is about 47 nM. In some embodiments, the single chain variable fragment CD3 binding protein has an amino acid sequence set forth as SEQ ID NO. 18, wherein the hKd is about 16 nM, and wherein the cKd is about 16 nM. In some embodiments, the single chain variable fragment CD3 binding protein has an amino acid sequence set forth as SEQ ID NO. 19, wherein the hKd is about 46 nM, and wherein the cKd is about 43 nM. In some embodiments, the single chain variable fragment CD3 binding protein has an amino acid sequence set forth as SEQ ID NO. 20, wherein the hKd is about 18 nM, and wherein the cKd is about 17 nM. In some embodiments, the single chain variable fragment CD3 binding protein has an amino acid sequence set forth as SEQ ID NO. 21, wherein the hKd is about 133 nM, and wherein the cKd is about 134 nM. In some embodiments, the single chain variable fragment CD3 binding protein has an amino acid sequence set forth as SEQ ID NO. 94, wherein the hKd is about 117 nM, and wherein the cKd is about 115 nM. In some embodiments, the single chain variable fragment CD3 binding protein has an amino acid sequence set forth as SEQ ID NO. 95, wherein the hKd is about 109 nM, and wherein the cKd is about 103 nM.

In some embodiments, the single chain variable fragment CD3 binding protein has an amino acid sequence set forth as SEQ ID NO. 8, wherein the hKd and cKd are about the same as the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 9, wherein the hKd and cKd are about the same as the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 10, wherein the hKd and cKd are about the same as the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 11, wherein the hKd and cKd are about the same as the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 12, wherein the hKd and cKd are about the same as the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 13, wherein the hKd and cKd are about the same as the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 14, wherein the hKd and cKd are about 3-fold to about 5-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 15, wherein the hKd and cKd are about 3-fold to about 5-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 16, wherein the hKd and cKd are about 3-fold to about 5-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 17, wherein the hKd and cKd are about 6-fold to about 15-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 18, wherein the hKd and cKd are about 3-fold to about 5-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 19 (2A4), wherein the hKd and cKd are about 6-fold to about 15-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 20, wherein the hKd and cKd are about 3-fold to about 5-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 21, wherein the hKd and cKd are about 20-fold to about 50-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 94, wherein the hKd and cKd are about 20-fold to about 50-fold higher than the Kd towards CD3 of a protein which has the sequence as set forth in wt anti-CD3 (SEQ ID NO. 22). In some embodiments, the single chain variable fragment CD3 binding protein has an amino acid sequence set forth as SEQ ID NO. 95, wherein the hKd and cKd are about 20-fold to about 50-fold higher than the Kd towards CD3 of a protein which has the sequence as set forth in wt anti-CD3 (SEQ ID NO. 22).

### Half-Life Extension Domain

Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a decrease in in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect is provided a multispecific binding protein comprising a single chain variable fragment CD3 binding protein according to the present disclosure and further comprising a half-life extension domain, for example a domain which specifically binds to serum albumin. In some embodiments, the serum albumin binding domain of a single chain variable fragment CD3 binding protein can be any domain that binds to serum albumin including but not limited to domains from a monoclonal antibody, a polyclonal antibody, a recombinant antibody, a human antibody, a humanized antibody. In some embodiments, the serum albumin binding domain is a single chain variable fragments (scFv), single-domain antibody such as a heavy chain variable domain (VH), a light chain variable domain (VL) and a variable domain (VHH) of camelid derived sdAb, or antigen binding fragments of the HSA binding antibodies, such as Fab, Fab′, F(ab)2, and Fv fragments, fragments comprised of one or more CDRs, single-chain antibodies (e.g., single chain Fv fragments (scFv)), disulfide stabilized (dsFv) Fv fragments, heteroconjugate antibodies (e.g., bispecific antibodies), pFv fragments, heavy chain monomers or dimers, light chain monomers or dimers, and dimers consisting of one heavy chain and one light chain, peptide, ligand or small molecule entity specific for serum albumin. In certain embodiments, the HSA binding domain is a single-domain antibody. In other embodiments, the serum albumin binding domain is a peptide. In further embodiments, the serum albumin binding domain is a small molecule. It is contemplated that the serum albumin binding domain of the multispecific binding protein comprising a single chain variable fragment CD3 binding protein is fairly small and no more than 25 kD, no more than 20 kD, no more than 15 kD, or no more than 10 kD in some embodiments. In certain instances, the serum albumin binding is 5 kD or less if it is a peptide or small molecule entity.

The half-life extension domain of a multispecific binding protein comprising a single chain variable fragment CD3 binding protein provides for altered pharmacodynamics and pharmacokinetics of the single chain variable fragment CD3 binding protein itself. As above, the half-life extension domain extends the elimination half-time. The half-life extension domain also alters pharmacodynamic properties including alteration of tissue distribution, penetration, and diffusion of the single chain variable fragment CD3 binding protein. In some embodiments, the half-life extension domain provides for improved tissue (including tumor) targeting, tissue distribution, tissue penetration, diffusion within the tissue, and enhanced efficacy as compared with a protein without a half-life extension domain. In one embodiment, therapeutic methods effectively and efficiently utilize a reduced amount of the multispecific binding protein comprising a single chain variable fragment CD3 binding protein, resulting in reduced side effects, such as reduced non-tumor cell cytotoxicity.

Further, the binding affinity of the half-life extension domain can be selected so as to target a specific elimination half-time in a particular multispecific binding protein comprising a single chain variable fragment CD3 binding protein. Thus, in some embodiments, the half-life extension domain has a high binding affinity. In other embodiments, the half-life extension domain has a medium binding affinity. In yet other embodiments, the half-life extension domain has a low or marginal binding affinity. Exemplary binding affinities include Kd of 10 nM or less (high), between 10 nM and 100 nM (medium), and greater than 100 nM (low). As above, binding affinities to serum albumin are determined by known methods such as Surface Plasmon Resonance (SPR).

### Target Antigen Binding Domain

In addition to the described CD3 and half-life extension domains, the multispecific binding proteins described herein in certain embodiments also comprise a domain that binds to a target antigen. A target antigen is involved in and/or associated with a disease, disorder or condition. In particular, a target antigen associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell.

The design of the multispecific binding protein comprising a single chain variable fragment CD3 binding protein described herein allows the binding domain to a target antigen to be flexible in that the binding domain to a target antigen can be any type of binding domain, including but not limited to, domains from a monoclonal antibody, a polyclonal antibody, a recombinant antibody, a human antibody, a humanized antibody. In some embodiments, the binding domain to a target antigen is a single chain variable fragments (scFv), single-domain antibody such as a heavy chain variable domain (VH), a light chain variable domain (VL) and a variable domain (VHH) of camelid derived sdAb. In other embodiments, the binding domain to a target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to a target antigen is a ligand or peptide that binds to or associates with a target antigen. In yet further embodiments, the binding domain to a target antigen is a knottin. In yet further embodiments, the binding domain to a target antigen is a small molecular entity.

### Single Chain Variable Fragment CD3 Binding Protein Modifications

The single chain variable fragment CD3 binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in single chain variable fragment CD3 binding proteins described herein, including the peptide backbone, the amino acid side-chains, and the amino or carboxyl termini. Certain common peptide modifications that are useful for modification of single chain variable fragment CD3 binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

### Polynucleotides Encoding Single Chain Variable Fragment CD3 Binding Proteins

Also provided, in some embodiments, are polynucleotide molecules encoding a single chain variable fragment CD3 binding protein or a multispecific binding protein comprising a single chain variable fragment CD3 binding protein according to the present disclosure. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the three binding domains either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. In the embodiments where the target antigen binding domain is a small molecule, the polynucleotides contain genes encoding the CD3 binding domain and the half-life extension domain. In the embodiments where the half-life extension domain is a small molecule, the polynucleotides contain genes encoding the domains that bind to CD3 and the target antigen. Depending on the vector system and host utilized, any number of suitable transcription and translation elements, including constitutive and inducible promoters, may be used. The promoter is selected such that it drives the expression of the polynucleotide in the respective host cell.

In some embodiments, the polynucleotide is inserted into a vector, preferably an expression vector, which represents a further embodiment. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described single chain variable fragment CD3 binding protein. Examples of expression vectors for expression in E. coli are pSKK (Le Gall et al., J Immunol Methods. (2004) 285(1):111-27) or pcDNA5 (Invitrogen) for expression in mammalian cells, PICHIAPINK™ Yeast Expression Systems (Invitrogen), BACUVANCE™ Baculovirus Expression System (GenScript).

Thus, the single chain variable fragment CD3 binding proteins as described herein, in some embodiments, are produced by introducing a vector encoding the protein as described above into a host cell and culturing said host cell under conditions whereby the protein domains are expressed, may be isolated and, optionally, further purified.

### Production of Single Chain Variable Fragment CD3 Binding Proteins

Disclosed herein, in some embodiments, is a process for the production of a single chain variable fragment CD3 binding protein. In some embodiments, the process comprises culturing a host transformed or transfected with a vector comprising a nucleic acid sequence encoding a single chain variable fragment CD3 binding protein under conditions allowing the expression of the single chain variable fragment CD3 binding protein and recovering and purifying the produced protein from the culture.

In additional embodiment is provided a process directed to improving one or more properties, e.g. affinity, stability, heat tolerance, cross-reactivity, etc., of the single chain variable fragment CD3 binding protein and/or the multispecific binding proteins comprising a single chain variable fragment CD3 binding protein as described herein, compared to a reference binding compound. In some embodiments, a plurality of single-substitution libraries is provided each corresponding to a different domain, or amino acid segment of the single chain variable fragment CD3 binding protein or reference binding compound such that each member of the single-substitution library encodes only a single amino acid change in its corresponding domain, or amino acid segment. (This allows all of the potential substitutions in a large protein or protein binding site to be probed with a few small libraries.) In some embodiments, the plurality of domains forms or covers a contiguous sequence of amino acids of the single chain variable fragment CD3 binding protein or a reference binding compound. Nucleotide sequences of different single-substitution libraries overlap with the nucleotide sequences of at least one other single-substitution library. In some embodiments, a plurality of single-substitution libraries are designed so that every member overlaps every member of each single-substitution library encoding an adjacent domain.

Binding compounds expressed from such single-substitution libraries are separately selected to obtain a subset of variants in each library which has properties at least as good as those of the reference binding compound and whose resultant library is reduced in size. (That is, the number of nucleic acids encoding the selected set of binding compounds is smaller than the number of nucleic acids encoding members of the original single-substitution library) Such properties include, but are not limited to, affinity to a target compound, stability with respect to various conditions such as heat, high or low pH, enzymatic degradation, cross-reactivity to other proteins and the like. The selected compounds from each single-substitution library are referred to herein interchangeably as “pre-candidate compounds,” or “pre-candidate proteins.” Nucleic acid sequences encoding the pre-candidate compounds from the separate single-substitution libraries are then shuffled in a PCR to generate a shuffled library, using PCR-based gene shuffling techniques.

An exemplary work flow of the screening process is described herein. Libraries of pre-candidate compounds are generated from single substitution libraries and selected for binding to the target protein(s), after which the pre-candidate libraries are shuffled to produce a library of nucleic acids encoding candidate compounds which, in turn, are cloned into a convenient expression vector, such as a phagemid expression system. Phage expressing candidate compounds then undergo one or more rounds of selection for improvements in desired properties, such as binding affinity to a target molecule. Target molecules may be adsorbed or otherwise attached to a surface of a well or other reaction container, or target molecules may be derivatized with a binding moiety, such as biotin, which after incubation with candidate binding compounds may be captured with a complementary moiety, such as streptavidin, bound to beads, such as magnetic beads, for washing. In exemplary selection regimens, the candidate binding compounds undergo a prolonged wash step so that only candidate compounds with very low dissociation rates from a target molecule are selected. Exemplary wash times for such embodiments are at least 8 hours; or in other embodiments, at least 24 hours; or in other embodiments, at least 48 hours; or in other embodiments, at least 72 hours. Isolated clones after selection are amplified and subjected to an additional cycle of selection or analyzed, for example by sequencing and by making comparative measurements of binding affinity, for example, by ELISA, surface plasmon resonance binding, bio-layer interferometry (e.g. Octet system, ForteBio, Menlo Park, CA) or the like.

In some embodiments, the process is implemented to identify one or more single chain variable fragment CD3 binding protein and/or a multispecific binding protein comprising a single chain variable fragment CD3 binding protein with improved thermal stability, improved cross reactivity to a selected set of binding targets compared to that of a reference CD3 binding protein, such as a protein having the amino acid sequence of SEQ ID NO. 22. Single substitution libraries are prepared by varying codons in the VH and VL regions of the reference CD3 binding protein, including both codons in framework regions and in CDRs; in another embodiment, the locations where codons are varied comprise the CDRs of the heavy and light chains of the reference CD3 binding protein, or a subset of such CDRs, such as solely CDR1, solely CDR2, solely CDR3, or pairs thereof. In another embodiment, locations where codons are varied occur solely in framework regions. In some embodiments, a library comprises single codon changes solely from a reference CD3 binding protein solely in framework regions of both VH and VL numbering in the range of from 10 to 250. In another embodiment, the locations where codons are varied comprise the CDR3s of the heavy and light chains of the reference CD3 binding protein, or a subset of such CDR3s. In another embodiment, the number of locations where codons of VH and VL encoding regions are varied are in the range of from 10 to 250, such that up to 100 locations are in framework region. After preparation of the single substitution library, as outlined above, the following steps are carried out: (a) expressing separately each member of each single substitution library as a pre-candidate protein; (b) selecting members of each single substitution library which encode pre-candidate proteins which bind to a binding partner that differs from the original binding target [e.g. a desired cross-reaction target(s)]; (c) shuffling members of the selected libraries in a PCR to produce a combinatorial shuffled library; (d) expressing members of the shuffled library as candidate CD3 binding proteins; and (e) selecting members of the shuffled library one or more times for candidate CD3 binding proteins which bind the original binding partner and (f) further selecting the candidate proteins for binding to the desired cross-reactive target(s) thereby providing a nucleic acid encoded CD3 binding protein with increased cross reactivity for the one or more substances with respect to the reference CD3 binding protein without loss of affinity for the original ligand. In additional embodiments, the method may be implemented for obtaining a single chain variable fragment CD3 binding protein with decreased reactivity to a selected cross-reactive substance(s) or compound(s) or epitope(s) by substituting step (f) with the following step: depleting candidate binding compounds one or more times from the subset of candidate single chain variable fragment CD3 binding protein which bind to the undesired cross-reactive compound.

### Pharmaceutical Compositions

Also provided, in some embodiments, are pharmaceutical compositions comprising a single chain variable fragment CD3 binding protein described herein, a vector comprising the polynucleotide encoding the polypeptide of the single chain variable fragment CD3 binding protein or a host cell transformed by this vector and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

In some embodiments of the pharmaceutical compositions, the single chain variable fragment CD3 binding protein described herein is encapsulated in nanoparticles. In some embodiments, the nanoparticles are fullerenes, liquid crystals, liposome, quantum dots, superparamagnetic nanoparticles, dendrimers, or nanorods. In other embodiments of the pharmaceutical compositions, the single chain variable fragment CD3 binding protein is attached to liposomes. In some instances, the single chain variable fragment CD3 binding protein are conjugated to the surface of liposomes. In some instances, the single chain variable fragment CD3 binding protein are encapsulated within the shell of a liposome. In some instances, the liposome is a cationic liposome.

The single chain variable fragment CD3 binding proteins described herein are contemplated for use as a medicament. Administration is effected by different ways, e.g. by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a single chain variable fragment CD3 binding protein described herein. In some instances, the administration of a single chain variable fragment CD3 binding protein described herein induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue.

Also provided herein are methods and uses for a treatment of a disease, disorder or condition associated with a target antigen comprising administering to an individual in need thereof a single chain variable fragment CD3 binding protein described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In one embodiment, the disease, disorder or condition associated with a target antigen is cancer. In one instance, the cancer is a hematological cancer. In another instance, the cancer is a solid tumor cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the single chain variable fragment CD3 binding proteins are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the single chain variable fragment CD3 binding proteins are administered in combination with anti-diarrheal agents, anti-emetic agents, analgesics, opioids and/or non-steroidal anti-inflammatory agents. In some embodiments, the single chain variable fragment CD3 binding proteins are administered before, during, or after surgery.

### EXAMPLES

### Example 1: Identification of Anti-CD3 scFv Variants with Varying Affinities for Human CD3ε

Characterization of Parental anti-CD3E Phage

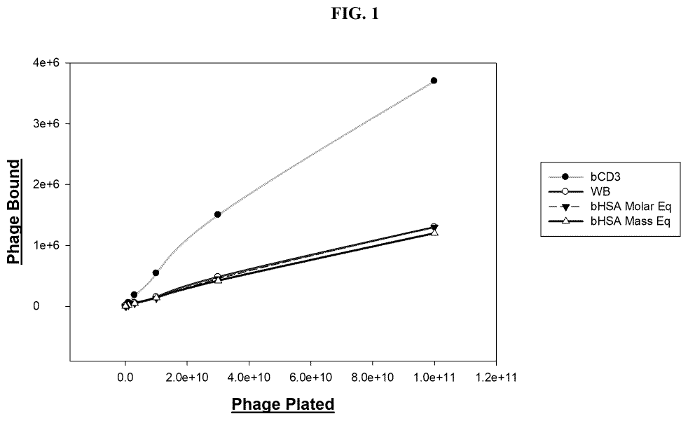

The parental anti-CD3ε showed good binding to biotin-CD3ε and low binding to biotin-HSA (FIG. 1).

Anti-CD3E scFv Phage Libraries

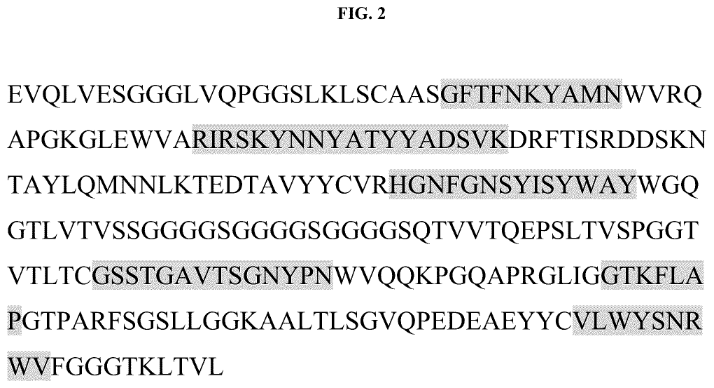

A single substitution library was provided for the heavy chain CDR1, heavy chain CDR2, heavy chain CDR3, light chain CDR1, light chain CDR2, and light chain CDR3 domains. The amino acid residues varied in each domain are illustrated in the highlighted region in FIG. 2. Residues were varied one at a time via NNN mutagensis.

### Selection of Clones and Determination of Binding Affinity

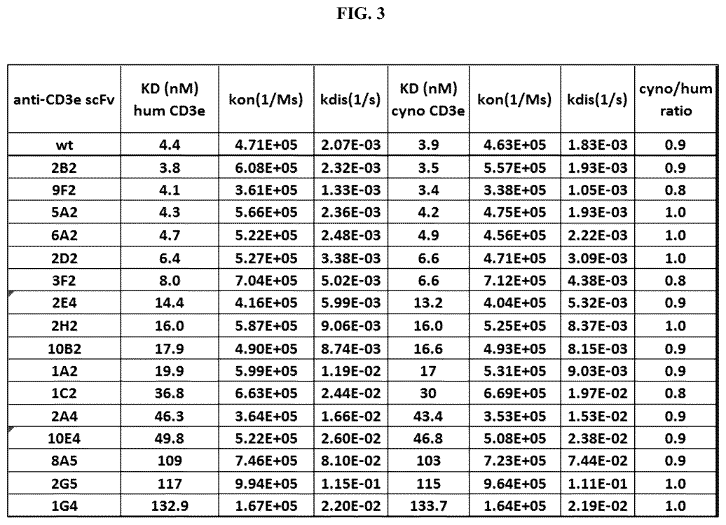

Single substitution libraries were bound to biotinylated hu-CD3ε, washed, eluted, and counted. Biotinylated cynoCD3 was used as the round1 selection target, and washed for 4 hours after combinatorial phage binding from the two independent libraries (˜2x selection). Biotinylated hu-CD3 was used as the round 2 selection target, and washed for 3 hours after binding of both libraries (<2x selection). PCRed inserts from the second round of selection were subcloned into the pcDNA3.4 His6 expression vector (“His6” disclosed as SEQ ID NO: 105). 180 clones were picked and DNA was purified, sequenced, and transfected into Expi293. A panel of sixteen clones with a range of affinities for human CD3ε were selected for more precise K_{d} determination (FIG. 3).

### Example 2: Cytotoxicity Assay

A Bispecific Antibody Directed to CD20 and CD3, Containing an Anti-CD3 scFv Variant Identified in Example 1 is Evaluated In Vitro on its Mediation of T Cell Dependent Cytotoxicity to CD20⁺ Target Cells.

Fluorescence labeled CD20⁺REC-1 cells (a Mantle cell lymphoma cell line, ATCC CRL-3004) are incubated with isolated PBMC of random donors or CB15 T-cells (standardized T-cell line) as effector cells in the presence of the CD20-CD3 bispecific antibody containing an anti-CD3 scFv variant identified in Example 1. After incubation for 4 h at 37° C. in a humidified incubator, the release of the fluorescent dye from the target cells into the supernatant is determined in a spectrofluorimeter. Target cells incubated without the CD20-CD3 bispecific antibody containing an anti-CD3 scFv variant identified in Example 1 and target cells totally lysed by the addition of saponin at the end of the incubation serve as negative and positive controls, respectively.

Based on the measured remaining living target cells, the percentage of specific cell lysis is calculated according to the following formula: [1-(number of living targets₍ₛₐₘₚₗₑ₎/number of living targets(spontaneous)]×100%. Sigmoidal dose response curves and EC₅₀ values are calculated by non-linear regression/4-parameter logistic fit using the GraphPad Software. The lysis values obtained for a given variant concentration are used to calculate sigmoidal dose-response curves by 4 parameter logistic fit analysis using the Prism software.

### Example 3: Thermal Stability of Anti-CD3 scFv Variants with Varying Affinities for Human CD3ε

The temperature of hydrophobic exposure (T_{b}) of a protein corresponds to the derivative of the inflection point of peak dye fluorescence and is known to correlate with melting temperature (Tₘ), which is a measure of protein stability. The goal of this study was to assess the Tₕ for several anti-human CD3ε scFv variants.

### Protein Production

Sequences of anti-human CD3ε scFv binding domains were cloned into pcDNA3.4 (Invitrogen) preceded by a leader sequence and followed by a 6x Histidine tag (SEQ ID NO: 105). Expi293F cells (Life Technologies A14527) were maintained in suspension in Optimum Growth Flasks (Thomson) between 0.2 to 8×1e6 cells/mL in Expi 293 media. Purified plasmid DNA was transfected into Expi293F cells in accordance with Expi293 Expression System Kit (Life Technologies, A14635) protocols, and maintained for 4-6 days post transfection. Conditioned media was partially purified by affinity and desalting chromatography. Anti-human CD3g scFv proteins were concentrated with Amicon Ultra centrifugal filtration units (EMD Millipore), applied to Superdex 200 size exclusion media (GE Healthcare) and resolved in a neutral buffer containing excipients. Fraction pooling and final purity were assessed by SDS-PAGE and analytical SEC (size exclusion chromatography). The absorbance of purified protein solutions were determined at 280 nm using a SpectraMax M2 (Molecular Devices) and UV-transparent 96-well plates (Corning 3635) and their concentrations were calculated from molar extinction coefficients.

### Differential Scanning Fluorimetry

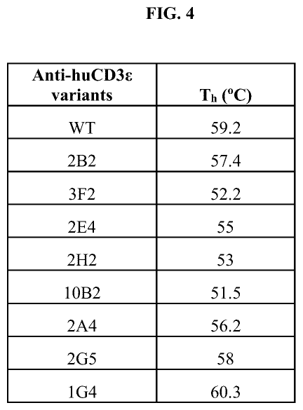

Purified anti-human CD3ε scFv proteins were diluted to cocentrations ranging from 0.2 to 0.25 mg/mL together with 5x SYPRO orange dye (Life Technologies 56651) in 0.15% DMSO final concentration in a neutral buffer containing excipients into MicroAmp EnduraPlate optical microplates and adhesive film (Applied Biosystems 4483485 and 4311971). A plate containing diluted protein and dye mixtures was loaded into an ABI 7500 Fast real-time PCR instrument (Applied Biosytems) and subjected to a multi-step thermal gradient from 25° C. to 95° C. The thermal gradient comprised of a two minute hold at each one degree celsius step with excitation at 500 nm and emission collected with a ROX filter. Tₕ in degrees celsius is presented, for several purified anti-human CD3ε scFv protein variants, in FIG. 4.

| SEQ ID | | |
|---|---|---|
| NO: | Description | AA Sequence |
| 1 | Linker | GGGGSGGGGSGGGGS |
| 2 | HC CDR1 with valiant | GX₁X₂X₃NX₄YX₅X₆N |
| | positions | |
| 3 | HC CDR2 with variant | R I R S X₇ X₈ N X₉ Y X₁₀ T X₁₁ Y X₁₂ D X₁₃ V K |
| | positions | |
| 4 | HC CDR3 with variant | H X₁₄ N F X₁₅ X₁₆ S X₁₇ I S Y W A X₁₈ |
| | positions | |
| 5 | LC CDR1 with variant | X₁₉ X₂₀ X₂₁ X₂₂ G X₂₃ V X₂₄ X₂₅ G X₂₆ Y P N |
| | positions | |
| 6 | LC CDR2 with variant | G X₂₇ X₂₈ X₂₉ X₃₀ X₃₁ P |
| | positions | |
| 7 | LC CDR3 with variant | X₃₂ L W Y X₃₃ N X₃₄ W X₃₅ |
| | positions | |
| 8 | Anti-CD3, clone 2B2 | EVQLVESGGGLVQPGGSLKLSCAASGFTFNKYAINWVRQAPGKGL |
| | | EWVARIRSKYNNYATYYADQVKDRFTISRDDSKNTAYLQMNNLKT |
| | | EDTAVYYCVRHANFGNSYISYWAYWGQGTLVTVSSGGGGSGGGG |
| | | SGGGGSQTVVTQEPSLTVSPGGTVTLTCASSTGAVTSGNYPNWVQQ |
| | | KPGQAPRGLIGGTKFLVPGTPARFSGSLLGGKAALTLSGVQPEDEA |
| | | EYYCTLWYSNRWVFGGGTKLTVL |
| 9 | Anti-CD3, clone 9F2 | EVQLVESGGGLVQPGGSLKLSCAASGFEFNKYAMNWVRQAPGKG |
| | | LEWVARIRSKYNKYATYYADSVKDRFTISRDDSKNTAYLQMNNLK |
| | | TEDTAVYYCVRHGNFGNSYISYWAYWGQGTLVTVSSGGGGSGGG |
| | | GSGGGGSQTVVTQEPSLTVSPGGTVTLTCGSSFGAVTSGNYPNWVQ |
| | | QKPGQAPRGLIGGTKFLAPGTPARFSGSLLGGKAALTLSGVQPEDE |
| | | AEYYCVLWYDNRWVFGGGTKLTVL |
| 10 | Anti-CD3, clone 5A2 | EVQLVESGGGLVQPGGSLKLSCAASGFTFNKYAMNWVRQAPGKG |
| | | LEWVARIRSKYNNYATYYADSVKDRFTISRDDSKNTAYLQMNNLK |
| | | TEDTAVYYCVRHGNFGNSHISYWAYWGQGTLVTVSSGGGGSGGG |
| | | GSGGGGSQTVVTQEPSLTVSPGGTVTLTCGSSTGYVTSGNYPNWVQ |
| | | QKPGQAPRGLIGGTSFLAPGTPARFSGSLLGGKAALTLSGVQPEDE |
| | | AEYYCVLWYSNRWIFGGGTKLTVL |
| 11 | Anti-CD3, clone 6A2 | EVQLVESGGGLVQPGGSLKLSCAASGFMFNKYAMNWVRQAPGKG |
| | | LEWVARIRSKSNNYATYYADSVKDRFTISRDDSKNTAYLQMNNLK |
| | | TEDTAVYYCVRHGNFGNSYISYWATWGQGTLVTVSSGGGGSGGG |
| | | GSGGGGSQTVVTQEPSLTVSPGGTVTLTCGSSFGAVTSGNYPNWVQ |
| | | QKPGQAPRGLIGGTKLLAPGTPARFSGSLLGGKAALTLSGVQPEDE |
| | | AEYYCVLWYSNSWVFGGGTKLTVL |
| 12 | Anti-CD3, clone 2D2 | EVQLVESGGGLVQPGGSLKLSCAASGFTFNTYAMNWVRQAPGKGL |
| | | EWVARIRSKYNNYATYYKDSVKDRFTISRDDSKNTAYLQMNNLKT |
| | | EDTAVYYCVRHGNFGNSPISYWAYWGQGTLVTVSSGGGGSGGGG |
| | | SGGGGSQTVVTQEPSLTVSPGGTVTLTCGSSTGAVVSGNYPNWVQ |
| | | QKPGQAPRGLIGGTEFLAPGTPARFSGSLLGGKAALTLSGVQPEDE |
| | | AEYYCVLWYSNRWVFGGGTKLTVL |
| 13 | Anti-CD3, clone 3F2 | EVQLVESGGGLVQPGGSLKLSCAASGFTYNKYAMNWVRQAPGKG |
| | | LEWVARIRSKYNNYATYYADEVKDRFTISRDDSKNTAYLQMNNLK |
| | | TEDTAVYYCVRHGNFGNSPISYWAYWGQGTLVTVSSGGGGSGGG |
| | | GSGGGGSQTVVTQEPSLTVSPGGTVTLTCGSSKGAVTSGNYPNWV |
| | | QQKPGQAPRGLIGGTKELAPGTPARFSGSLLGGKAALTLSGVQPED |
| | | EAEYYCTLWYSNRWVFGGGTKLTVL |
| 14 | Anti-CD3, clone 1A2 | EVQLVESGGGLVQPGGSLKLSCAASGNTFNKYAMNWVRQAPGKG |
| | | LEWVARIRSKYNNYETYYADSVKDRFTISRDDSKNTAYLQMNNLK |
| | | TEDTAVYYCVRHTNFGNSYISYWAYWGQGTLVTVSSGGGGSGGG |
| | | GSGGGGSQTVVTQEPSLTVSPGGTVTLTCGSSTGAVTSGYYPNWVQ |
| | | QKPGQAPRGLIGGTYFLAPGTPARFSGSLLGGKAALTLSGVQPEDE |
| | | AEYYCVLWYSNRWVFGGGTKLTVL |
| 15 | Anti-CD3, clone 1C2 | EVQLVESGGGLVQPGGSLKLSCAASGFTFNNYAMNWVRQAPGKG |
| | | LEWVARIRSKYNNYATYYADAVKDRFTISRDDSKNTAYLQMNNLK |
| | | TEDTAVYYCVRHGNFGNSQISYWAYWGQGTLVTVSSGGGGSGGG |
| | | GSGGGGSQTVVTQEPSLTVSPGGTVTLTCGSSTGAVTDGNYPNWV |
| | | QQKPGQAPRGLIGGIKFLAPGTPARFSGSLLGGKAALTLSGVQPED |
| | | EAEYYCVLWYSNRWVFGGGTKLTVL |
| 16 | Anti-CD3, clone 2E4 | EVQLVESGGGLVQPGGSLKLSCAASGFTFNKYAVNWVRQAPGKGL |
| | | EWVARIRSKYNNYATYYADSVKDRFTISRDDSKNTAYLQMNNLKT |
| | | EDTAVYYCVRHGNFGNSYISYWAYWGQGTLVTVSSGGGGSGGGG |
| | | SGGGGSQTVVTQEPSLTVSPGGTVTLTCGESTGAVTSGNYPNWVQ |
| | | QKPGQAPRGLIGGTKILAPGTPARFSGSLLGGKAALTLSGVQPEDE |
| | | AEYYCVLWYSNRWVFGGGTKLTVL |
| 17 | Anti-CD3, clone 10E4 | EVQLVESGGGLVQPGGSLKLSCAASGFTFNKYPMNWVRQAPGKGL |
| | | EWVARIRSKYNNYATYYADSVKDRFTISRDDSKNTAYLQMNNLKN |
| | | EDTAVYYCVRHGNFNNSYISYWAYWGQGTLVTVSSGGGGSGGGG |
| | | SGGGGSQTVVTQEPSLTVSPGGTVTLTCGSSTGAVTKGNYPNWVQ |
| | | QKPGQAPRGLIGGTKMLAPGTPARFSGSLLGGKAALTLSGVQPEDE |
| | | AEYYCALWYSNRWVFGGGTKLTVL |
| 18 | Anti-CD3, clone 2H2 | EVQLVESGGGLVQPGGSLKLSCAASGFTFNGYAMNWVRQAPGKG |
| | | LEWVARIRSKYNNYATYYADEVKDRFTISRDDSKNTAYLQMNNLK |
| | | TEDTAVYYCVRHGNFGNSPISYWAYWGQGTLVTVSSGGGGSGGG |
| | | GSGGGGSQTVVTQEPSLTVSPGGTVTLTCGSSTGAVVSGNYPNWV |
| | | QQKPGQAPRGLIGGTEFLAPGTPARFSGSLLGGKAALTLSGVQPED |
| | | EAEYYCVLWYSNRWVFGGGTKLTVL |
| 19 | Anti-CD3, clone 2A4 | EVQLVESGGGLVQPGGSLKLSCAASGNTFNKYAMNWVRQAPGKG |
| | | LEWVARIRSKYNNYATYYADSVKDRFTISRDDSKNTAYLQMNNLK |
| | | TEDTAVYYCVRHGNFGDSYISYWAYWGQGTLVTVSSGGGGSGGG |
| | | GSGGGGSQTVVTQEPSLTVSPGGTVTLTCGSSTGAVTHGNYPNWV |
| | | QQKPGQAPRGLIGGTKVLAPGTPARFSGSLLGGKAALTLSGVQPED |
| | | EAEYYCVLWYSNRWVFGGGTKLTVL |
| 20 | Anti-CD3, clone 10B2 | EVQLVESGGGLVQPGGSLKLSCAASGFTFNNYAMNWVRQAPGKG |
| | | LEWVARIRSGYNNYATYYADSVKDRFTISRDDSKNTAYLQMNNLK |
| | | TEDTAVYYCVRHGNFGNSYISYWAYWGQGTLVTVSSGGGGSGGG |
| | | GSGGGGSQTVVTQEPSLTVSPGGTVTLTCGSYTGAVTSGNYPNWV |
| | | QQKPGQAPRGLIGGTKFNAPGTPARFSGSLLGGKAALTLSGVQPED |
| | | EAEYYCVLWYANRWVFGGGTKLTVL |
| 21 | Anti-CD3, clone 1G4 | EVQLVESGGGLVQPGGSLKLSCAASGFEFNKYAMNWVRQAPGKG |
| | | LEWVARIRSKYNNYETYYADSVKDRFTISRDDSKNTAYLQMNNLK |
| | | TEDTAVYYCVRHGNFGNSLISYWAYWGQGTLVTVSSGGGGSGGG |
| | | GSGGGGSQTVVTQEPSLTVSPGGTVTLTCGSSSGAVTSGNYPNWVQ |
| | | QKPGQAPRGLIGGTKFGAPGTPARFSGSLLGGKAALTLSGVQPEDE |
| | | AEYYCVLWYSNRWVFGGGTKLTVL |
| 22 | wt anti-CD3 | EVQLVESGGGLVQPGGSLKLSCAASGFTFNKYAMNWVRQAPGKG |
| | | LEWVARIRSKYNNYATYYADSVKDRFTISRDDSKNTAYLQMNNLK |
| | | TEDTAVYYCVRHGNFGNSYISYWAYWGQGTLVTVSSGGGGSGGG |
| | | GSGGGGSQTVVTQEPSLTVSPGGTVTLTCGSSTGAVTSGNYPNWVQ |
| | | QKPGQAPRGLIGGTKFLAPGTPARFSGSLLGGKAALTLSGVQPEDE |
| | | AEYYCVLWYSNRWVFGGGTKLTVL |
| 23 | wt anti-CD3 HC CDR1 | GFTFNKYAMN |
| 24 | wt anti-CD3 HC CDR2 | RIRSKYNNYATYYADSVK |
| 25 | wt anti-CD3 HC CDR3 | HGNFGNSYISYWAY |
| 26 | wt anti-CD3 LC CDR1 | GSSTGAVTSGNYPN |
| 27 | wt anti-CD3 LC CDR2 | GTKFLAP |
| 28 | wt anti-CD3 LC CDR3 | VLWYSNRWV |
| 29 | HC CDR1 variant 1 | GNTFNKYAMN |
| 30 | HC CDR1 variant 2 | GFEFNKYAMN |
| 31 | HC CDR1 variant 3 | GFMFNKYAMN |
| 32 | HC CDR1 variant 4 | GFTYNKYAMN |
| 33 | HC CDR1 variant 5 | GFTFNNYAMN |
| 34 | HC CDR1 variant 6 | GFTFNGYAMN |
| 35 | HC CDR1 variant 7 | GFTFNTYAMN |
| 36 | HC CDR1 variant 8 | GFTFNEYAMN |
| 37 | HC CDR1 variant 9 | GFTFNKYPMN |
| 38 | HC CDR1 variant 10 | GFTFNKYAVN |
| 39 | HC CDR1 variant 11 | GFTFNKYAIN |
| 40 | HC CDR1 variant 12 | GFTFNKYALN |
| 41 | HC CDR2 variant 1 | RIRSGYNNYATYYADSVK |
| 42 | HC CDR2 variant 2 | RIRSKSNNYATYYADSVK |
| 43 | HC CDR2 variant 3 | RIRSKYNKYATYYADSVK |
| 44 | HC CDR2 variant 4 | RIRSKYNNYETYYADSVK |
| 45 | HC CDR2 variant 5 | RIRSKYNNYATEYADSVK |
| 46 | HC CDR2 variant 6 | RIRSKYNNYATYYKDSVK |
| 47 | HC CDR2 variant 7 | RIRSKYNNYATYYADEVK |
| 48 | HC CDR2 variant 8 | RIRSKYNNYATYYADAVK |
| 49 | HC CDR2 variant 9 | RIRSKYNNYATYYADQVK |
| 50 | HC CDR2 variant 10 | RIRSKYNNYATYYADDVK |
| 51 | HC CDR3 variant 1 | HANFGNSYISYWAY |
| 52 | HC CDR3 variant 2 | HTNFGNSYISYWAY |
| 53 | HC CDR3 variant 3 | HGNFNNSYISYWAY |
| 54 | HC CDR3 variant 4 | HGNFGDSYISYWAY |
| 55 | HC CDR3 variant 5 | HGNFGNSHISYWAY |
| 56 | HC CDR3 variant 6 | HGNFGNSPISYWAY |
| 57 | HC CDR3 variant 7 | HGNFGNSQISYWAY |
| 58 | HC CDR3 variant 8 | HGNFGNSLISYWAY |
| 59 | HC CDR3 variant 9 | HGNFGNSGISYWAY |
| 60 | HC CDR3 variant 10 | HGNFGNSYISYWAT |
| 61 | LC CDR1 variant 1 | ASSTGAVTSGNYPN |
| 62 | LC CDR1 variant 2 | GESTGAVTSGNYPN |
| 63 | LC CDR1 variant 3 | GSYTGAVTSGNYPN |
| 64 | LC CDR1 variant 4 | GSSFGAVTSGNYPN |
| 65 | LC CDR1 variant 5 | GSSKGAVTSGNYPN |
| 66 | LC CDR1 variant 6 | GSSSGAVTSGNYPN |
| 67 | LC CDR1 variant 7 | GSSTGYVTSGNYPN |
| 68 | LC CDR1 variant 8 | GSSTGAVVSGNYPN |
| 69 | LC CDR1 variant 9 | GSSTGAVTDGNYPN |
| 70 | LC CDR1 variant 10 | GSSTGAVTKGNYPN |
| 71 | LC CDR1 variant 11 | GSSTGAVTHGNYPN |
| 72 | LC CDR1 variant 12 | GSSTGAVTVGNYPN |
| 73 | LC CDR1 variant 13 | GSSTGAVTSGYYPN |
| 74 | LC CDR2 variant 1 | GIKFLAP |
| 75 | LC CDR2 variant 2 | GTEFLAP |
| 76 | LC CDR2 variant 3 | GTYFLAP |
| 77 | LC CDR2 variant 4 | GTSFLAP |
| 78 | LC CDR2 variant 5 | GTNFLAP |
| 79 | LC CDR2 variant 6 | GTKLLAP |
| 80 | LC CDR2 variant 7 | GTKELAP |
| 81 | LC CDR2 variant 8 | GTKILAP |
| 82 | LC CDR2 variant 9 | GTKMLAP |
| 83 | LC CDR2 variant 10 | GTKVLAP |
| 84 | LC CDR2 variant 11 | GTKFNAP |
| 85 | LC CDR2 variant 12 | GTKFGAP |
| 86 | LC CDR2 variant 13 | GTKFLVP |
| 87 | LC CDR3 variant 1 | TLWYSNRWV |
| 88 | LC CDR3 variant 2 | ALWYSNRWV |
| 89 | LC CDR3 variant 3 | VLWYDNRWV |
| 90 | LC CDR3 variant 4 | VLWYANRWV |
| 91 | LC CDR3 variant 5 | VLWYSNSWV |
| 92 | LC CDR3 variant 6 | VLWYSNRWI |
| 93 | LC CDR3 variant 7 | VLWYSNRWA |
| 94 | Anti-CD3, clone 2G5 | EVQLVESGGGLVQPGGSLKLSCAASGFTFNKYALNWVRQAPGKGL |
| | | EWVARIRSKYNNYATEYADSVKDRFTISRDDSKNTAYLQMNNLKT |
| | | EDTAVYYCVRHGNFGNSPISYWAYWGQGTLVTVSSGGGGSGGGG |
| | | SGGGGSQTVVTQEPSLTVSPGGTVTLTCGSSTGAVTSGNYPNWVQQ |
| | | KPGQAPRGLIGGTNFLAPGTPERFSGSLLGGKAALTLSGVQPEDEA |
| | | EYYCVLWYSNRWAFGGGTKLTVL |
| 95 | Anti-CD3, clone 8A5 | EVQLVESGGGLVQPGGSLKLSCAASGFTFNEYAMNWVRQAPGKGL |
| | | EWVARIRSKYNNYATYYADDVKDRFTISRDDSKNTAYLQMNNLKT |
| | | EDTAVYYCVRHGNFGNSGISYWAYWGQGTLVTVSSGGGGSGGGG |
| | | SGGGGSQTVVTQEPSLTVSPGGTVTLTCGSSTGAVTVGNYPNWVQ |
| | | QKPGQAPRGLIGGTEFLAPGTPARFSGSLLGGKAALTLSGVQPEDE |
| | | AEYYCVLWYSNRWVFGGGTKLTVL |
| 96 | Exemplary linker sequence | (GS)n |
| 97 | Exemplary linker sequence | (GGS)n |
| 98 | Exemplary linker sequence | (GGGS)n |
| 99 | Exemplary linker sequence | (GGSG)n |
| 100 | Exemplary linker sequence | (GGSGG)n |
| 101 | Exemplary linker sequence | (GGGGS)n |
| 102 | Exemplary linker sequence | (GGGGG)n |
| 103 | Exemplary linker sequence | (GGG)n |
| 104 | Exemplary linker sequence | (GGGGS)4 |
| 105 | 6X Histidine | HHHHHH |

1. A single chain variable fragment CD3 binding protein, comprising a variable heavy chain region (VH), a variable light chain region (VL), and a linker, wherein VH comprises complementarity determining regions HC CDR1, HC CDR2, and HC CDR3, wherein VL comprises complementarity determining regions LC CDR1, LC CDR2, and LC CDR3, wherein
(a) the amino acid sequence of HC CDR1 is as set forth in GX1X2X3NX4YX5X6N (SEQ ID NO. 2), X1 is phenylalanine or asparagine, X2 is threonine, glutamic acid or methionine, X3 is phenylalanine or tyrosine, X4 is lysine, threonine, glycine, asparagine or glutamic acid, X5 is alanine or proline, X6 is methionine, leucine, valine or isoleucine;
(b) the amino acid sequence of HC CDR2 is as set forth in RIRSX7X8NX9YX10TX11YX12DX13VK (SEQ ID NO. 3), X7 is lysine or glycine, X8 is tyrosine or serine, X9 is asparagine or lysine, X10 is alanine or glutamic acid, X11 is tyrosine or glutamic acid, X12 is alanine or lysine, X13 is serine, glutamic acid, aspartic acid, alanine, or glutamine;
(c) the amino acid sequence of HC CDR3 is as set forth in HX14NFX15 X16SX17ISYWAX18 (SEQ ID NO. 4), X14 is glycine, alanine, or threonine, X15 is glycine or asparagine, X16 is asparagine or aspartic acid, X17 is tyrosine, histidine, proline, glutamine, leucine or glycine, X18 is tyrosine or threonine;
(d) the amino acid sequence of LC CDRlis as set forth in X19X20X21X22GX23VX24X25GX26YPN (SEQ ID NO. 5), X19 is glycine or alanine, X20 is serine or glutamic acid, X21 is serine or tyrosine, X22 is threonine, phenylalanine, lysine, or serine, X23 is alanine or tyrosine, X24 is threonine or valine, X25 is serine, aspartic acid, lysine, histidine or valine, X26 asparagine or tyrosine;
(e) the amino acid sequence of LC CDR2is as set forth in GX27X28X29X30X31P÷SEQ ID NO. 6), X27 is threonine or isoleucine, X28 is lysine, glutamic acid, tyrosine, asparagine or serine, X29 is phenylalanine, leucine, glutamic acid, isoleucine, methionine, or valine, X30 is leucine, asparagine, or glycine, X31 is alanine or valine; and
(f) the amino acid sequence of LC CDR3is as set forth in X32LWYX33NX34WX35 (SEQ ID NO. 7), X32 is valine, threonine or alanine, X33 is serine, aspartic acid or alanine, X34 is arginine or serine, X35 is valine, isoleucine or alanine, wherein X1, X2, X3, X4, X5, X6, X7, X8, X9, X10, X11, X12, X13, X14, X15, X16, X17, X18, X19, X20, X21, X22, X23, X24, X25, X26, X27, X28, X29, X30, X31, X32, X33, X34 and X35 are not simultaneously phenylalanine, threonine, phenylalanine, lysine, alanine, methionine, lysine, tyrosine, asparagine, alanine, tyrosine, alanine, serine, glycine, glycine, asparagine, tyrosine, tyrosine, glycine, serine, serine, threonine, alanine, threonine, serine, asparagine, threonine, lysine, phenylalanine, leucine, alanine, valine, serine, arginine, and valine respectively.

2. The single chain variable fragment CD3 binding protein of claim 1, wherein said protein comprises the following formula:
f14- r1-f2-r2-f3-r3 -f4-r4-f5-r5- f6-r6-f7
wherein, r1 is SEQ ID NO: 2; r2 is SEQ ID NO: 3; r3 is SEQ ID NO: 4; r4 is SEQ ID NO:5; r5 is SEQ ID NO:6; and r6 is SEQ ID NO:7; and wherein f1, f2, f3, f4, and f5 are framework residues selected so that said protein is at least eighty percent identical to the amino acid sequence set forth in SEQ ID NO: 22.

3. The single chain variable fragment CD3 binding protein of claim 2, wherein r1 comprises SEQ ID NO. 29, SEQ ID NO. 30, SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO.38, SEQ ID NO. 39, or SEQ ID NO. 40.

4. The single chain variable fragment CD3 binding protein of claim 2, wherein r2 comprises SEQ ID NO. 41, SEQ ID NO. 42, SEQ ID NO. 43, SEQ ID NO. 44, SEQ ID NO. 45, SEQ ID NO. 46, SEQ ID NO. 47, SEQ ID NO. 48, SEQ ID NO. 49, or SEQ ID NO. 50.

5. The single chain variable fragment CD3 binding protein of claim 2, wherein r3 comprises SEQ ID NO. 51, SEQ ID NO. 52, SEQ ID NO. 53, SEQ ID NO. 54, SEQ ID NO. 55, SEQ ID NO. 56, SEQ ID NO. 57, SEQ ID NO. 58, SEQ ID NO. 50, or SEQ ID NO. 60.

6. The single chain variable fragment CD3 binding protein of claim 2, wherein r4 comprises SEQ ID NO. 61, SEQ ID NO. 62, SEQ ID NO. 63, SEQ ID NO. 64, SEQ ID NO. 65, SEQ ID NO. 66, SEQ ID NO. 67, SEQ ID NO. 68, SEQ ID NO. 69, SEQ ID NO. 70, SEQ ID NO. 71, SEQ ID NO. 72, or SEQ ID NO. 73.

7. The single chain variable fragment CD3 binding protein of claim 2, wherein r5 comprises SEQ ID NO. 74, SEQ ID NO. 75, SEQ ID NO. 76, SEQ ID NO. 77, SEQ ID NO. 78, SEQ ID NO. 79, SEQ ID NO. 80, SEQ ID NO. 81, SEQ ID NO. 82, SEQ ID NO. 83, SEQ ID NO. 84, SEQ ID NO. 85, or SEQ ID NO. 86.

8. The single chain variable fragment CD3 binding protein of claim 2, wherein r6 comprises SEQ ID NO. 87, SEQ ID NO. 88, SEQ ID NO. 89, SEQ ID NO. 90, SEQ ID NO. 91, SEQ ID NO. 92, or SEQ ID NO. 93.

9. The single chain variable fragment CD3 binding protein of claim 2, wherein r1 is SEQ ID NO. 39, r2 is SEQ ID NO. 49, r3 is SEQ ID NO. 51, r4 is SEQ ID NO. 61, r5 is SEQ ID NO. 86, and r6 is SEQ ID NO. 87.

10. The single chain variable fragment CD3 binding protein of claim 2, wherein r1 is SEQ ID NO. 30, r2 is SEQ ID NO. 43, r4 is SEQ ID NO. 64, and r6 is SEQ ID NO. 89.

11. The single chain variable fragment CD3 binding protein of claim 2, wherein r3 is SEQ ID NO. 55, r4 is SEQ ID NO. 67, r5 is SEQ ID NO. 77, and r6 is SEQ ID NO. 92.

12. The single chain variable fragment CD3 binding protein of claim 2, wherein r1 is SEQ ID NO. 31, r2 is SEQ ID NO. 42, r3 is SEQ ID NO. 60, r4 is SEQ ID NO. 64, r5 is SEQ ID NO. 79, and r6 is SEQ ID NO. 91.

13. The single chain variable fragment CD3 binding protein of claim 1, wherein r1 is SEQ ID NO. 35, r2 is SEQ ID NO. 46, r3 is SEQ ID NO. 56, r4 is SEQ ID NO. 68, and r5 is SEQ ID NO. 75. The single chain variable fragment CD3 binding protein of claim 1, wherein r1 is SEQ ID NO. 32, r2 is SEQ ID NO. 47, r3 is SEQ ID NO. 56, r4 is SEQ ID NO. 65, r5 is SEQ ID NO. 80, and r6 is SEQ ID NO. 87.

14. The single chain variable fragment CD3 binding protein of claim 2, wherein r1 is SEQ ID NO. 29, r2 is SEQ ID NO. 44, r3 is SEQ ID NO. 52, r4 is SEQ ID NO. 73, and r5 is SEQ ID NO. 76.

15. The single chain variable fragment CD3 binding protein of claim 2, wherein r1 is SEQ ID NO. 33, r2 is SEQ ID NO. 48, r3 is SEQ ID NO. 57, r4 is SEQ ID NO. 69, and r5 is SEQ ID NO. 74. The single chain variable fragment CD3 binding protein of claim 2, wherein r1 is SEQ ID NO. 38, r4 is SEQ ID NO. 62, and r5 is SEQ ID NO. 81.

16. The single chain variable fragment CD3 binding protein of claim 2, wherein r1 is SEQ ID NO. 37, r3 is SEQ ID NO. 53, r4 is SEQ ID NO. 70, r5 is SEQ ID NO. 82, and r6 is SEQ ID NO. 88.

17. The single chain variable fragment CD3 binding protein of claim 2, wherein r1 is SEQ ID NO. 34, r2 is SEQ ID NO. 47, r3 is SEQ ID NO. 56, r4 is SEQ ID NO. 68 and r5 is SEQ ID NO. 75.

18. The single chain variable fragment CD3 binding protein of claim 2, wherein r1 is SEQ ID NO. 29, r3 is SEQ ID NO. 54, r4 is SEQ ID NO. 71 and r5 is SEQ ID NO. 83.

19. The single chain variable fragment CD3 binding protein of claim 2, wherein r1 is SEQ ID NO. 33, r2 is SEQ ID NO. 41, r4 is SEQ ID NO. 63, r5 is SEQ ID NO. 84 and r6 is SEQ ID NO. 90. The single chain variable fragment CD3 binding protein of claim 2, wherein r1 is SEQ ID NO. 30, r2 is SEQ ID NO. 44, r3 is SEQ ID NO. 58, r4 is SEQ ID NO. 66 and r5 is SEQ ID NO. 85.

20. The single chain variable fragment CD3 binding protein of claim 2, wherein r1 is SEQ ID NO. 40, r2 is SEQ ID NO. 45, r3 is SEQ ID NO. 56, r5 is SEQ ID NO. 78 and r6 is SEQ ID NO. 93. The single chain variable fragment CD3 binding protein of claim 2, wherein r1 is SEQ ID NO. 36, r2 is SEQ ID NO. 50, r3 is SEQ ID NO. 59, r4 is SEQ ID NO. 72 and r5 is SEQ ID NO. 75.

21. The single chain variable fragment CD3 binding protein of claim 1, wherein said protein has an amino acid sequence selected from SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 94, and SEQ ID NO. 95.

22. (canceled)

23. (canceled)

24. (canceled)

25. (canceled)

26. (canceled)

27. A single chain variable fragment CD3 binding protein comprising the sequence set forth as SEQ ID NO. 22 (wt anti-CD3) wherein one or more amino acid residues selected from amino acid positions 27, 28, 29, 31, 33, 34, 54, 55, 57, 59, 61, 63, 65, 102, 105, 106, 108, 114, 163, 164, 165, 166, 168, 170, 171, 173, 193, 194, 195, 196, 197, 231, 235, 237, and 239 are substituted,
wherein
amino acid position 27 is substituted with asparagine,
amino acid position 28 is substituted with glutamic acid, or methionine,
amino acid position 29 is substituted with tyrosine,
amino acid position 31 is substituted with asparagine, glycine, glutamic acid or threonine,
amino acid position 33 is substituted with proline,
amino acid position 34 is substituted with valine, leucine or isoleucine,
amino acid position 54 is substituted with glycine,
amino acid position 55 is substituted with serine,
amino acid position 57 is substituted with lysine,
amino acid position 59 is substituted with glutamic acid,
amino acid position 61 is substituted with glutamic acid,
amino acid position 63 is substituted with lysine,
amino acid position 65 is substituted with aspartic acid, glutamic acid, alanine, or glutamine,
amino acid position 102 is substituted with alanine or threonine,
amino acid position 105 is substituted with asparagine,
amino acid position 106 is substituted with aspartic acid,
amino acid position 108 is substituted with histidine, proline, glutamine, glycine, or leucine,
amino acid position 114 is substituted with threonine,
amino acid position 163 is substituted with alanine,
amino acid position 164 is substituted with glutamic acid,
amino acid position 165 is substituted with tyrosine.
amino acid position 166 is substituted with phenylalanine, lysine, or serine,
amino acid position 168 is substituted with tyrosine,
amino acid position 170 is substituted with valine,
amino acid position 171 is substituted with aspartic acid, lysine, valine, or histidine,
amino acid position 173 is substituted with tyrosine,
amino acid position 193 is substituted with isoleucine,
amino acid position 194 is substituted with glutamic acid, tyrosine, asparagine, or serine,
amino acid position 195 is substituted with leucine, glutamic acid, isoleucine, methionine, or valine,
amino acid position 196 is substituted with asparagine, or glycine,
amino acid position 197 is substituted with valine,
amino acid position 231 is substituted with threonine, or alanine,
amino acid position 235 is substituted with aspartic acid, or alanine,
amino acid position 237 is substituted with serine, and
amino acid position 239 is substituted with alanine, or isoleucine.

28. (canceled)

29. A single chain variable fragment CD3 binding protein comprising an amino acid sequence as set forth in wt anti-CD3 (SEQ ID NO: 22), comprising a variable heavy chain region (VH), a variable light chain region (VL), a linker comprising the amino acid sequence as set forth in GGGGSGGGGSGGGGS (SEQ ID NO: 1), wherein VH comprises complementarity determining regions HC CDR1, HC CDR2, and HC CDR3, wherein VL comprises complementarity determining regions LC CDR1, LC CDR2, and LC CDR3, comprising at least one mutation in HC CDR1, HC CDR2 or HC CDR3 of VH, and LC CDR1, LC CDR2 or LC CDR3 of VL, wherein the at least one mutation is not in amino acid positions 26, 30, 32, 35, 50, 51, 52, 53, 56, 58, 60, 62, 64, 66, 67, 101, 103, 104, 107, 109, 110, 111, 112, 113, 167, 169, 172, 174, 175, 176, 192, 198, 232, 233, 234, 236, or 238.

30. (canceled)

31. A polynucleotide encoding a single chain variable fragment CD3 binding protein, wherein the protein has an amino acid sequence selected from SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 94, and SEQ ID NO. 95.

32. A vector comprising a polynucleotide, wherein the polynucleotide encodes a single chain variable fragment CD3 binding protein, wherein the protein has an amino acid sequence selected from SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 94, and SEQ ID NO. 95.

33. A host cell comprising (a) a polynucleotide encoding a single chain variable fragment CD3 binding protein, wherein the protein has an amino acid sequence selected from SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, SEQ ID NO. 94, and SEQ ID NO. 95, or (b) a vector comprising a polynucleotide of (a).

34. A method for treating a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease, comprising administering a single chain variable fragment CD3 binding protein of claim 1, a polynucleotide of claim 31, a vector of claim 32, or a host cell of claim 33, to a subject in need thereof.
